(19) United States
(12) Patent Application Publication
(10) Pub. No.: US 2010/0178245 A1
(43) Pub. Date: Jul. 15, 2010

(54) Biocompatible Microbubbles to Deliver Radioactive Compounds to Tumors, Atherosclerotic Plaques, Joints and Other Targeted Sites

(76) Inventors: Morton F. Arnsdorf, Beverly Shores, IN (US); Jenny L. Whitlock, Chicago, IL (US)

Correspondence Address: MCANDREWS HELD & MALLOY, LTD, 500 WEST MADISON STREET, SUITE 3400, CHICAGO, IL, 60661, US

(21) Appl. No.: 12/425,119

(22) Filed: Apr. 16, 2009

Related U.S. Application Data
(63) Continuation-in-part of application No. 12/352,740, filed on Jan. 13, 2009.

(51) Int. Cl. A61K 51/12; A61P 35/00

(52) U.S. Cl. 424/1.29

(57) ABSTRACT

A composition and method for targeted use of radionuclide therapy for the treatment of cancer and cancerous tumors, atherosclerotic plaques, joints and other targeted sites. Microparticles, microbubbles, or nanoparticles deliver therapeutic doses of radiation, included radiation from alpha emitting radionuclides, to sites in a patient. The delivery may be targeted by targeting agents linked to the microparticles, microbubbles, or nanoparticles or by the external application of energy, or both.

### RELATED APPLICATIONS

This is a continuation-in-part of U.S. patent application Ser. No. 12,352,740, filed on Jan. 13, 2009.

### BACKGROUND OF THE INVENTION

The present invention is generally directed to improvements in the use of radionuclide therapy for the treatment of cancer and cancerous tumors, atherosclerotic plaques, joints and other targeted sites.

Radiation therapy, also called radiotherapy, is the treatment of cancer and other diseases with ionizing radiation. Ionizing radiation deposits energy that injures or destroys cells in the area being treated (the target tissue) by damaging the genetic material (e.g., DNA) in the individual cells, making it impossible for them to continue to grow and in certain cases eventually killing them. The effects of radiation therapy are independent of oxygenation state or cell-cycle.

Although radiation damages both cancer cells and normal cells, normal, healthy cells are able to repair themselves and return to proper functioning. Radiotherapy has been used to treat localized solid tumors, such as those cancers associated with the oral environment. It has also been used to treat leukemia and lymphoma (cancers of the blood-forming cells and lymphatic system, respectively).

Ionizing radiation can be sorted into two major types: 1) photons (e.g., x-rays and gamma rays) and 2) particle radiation (e.g., electrons, protons, neutrons, alpha particles, and beta particles). Of the two types, photons are most widely used.

Some types of ionizing radiation have more energy than others. Generally, the higher the energy, the more deeply the radiation can penetrate the tissues, although some high intensity particles have a short range. The way a certain type of radiation behaves influences the planning of radiation treatments. A radiation oncologist typically selects the type and energy of radiation that is suitable for each patient's cancer.

Cancer patients are commonly treated with different types of radiation. For example, cancer patients can be treated with high-energy photons, electron beams, protons, or neutrons.

High-energy photons come from radioactive sources such as cobalt, cesium, or from a generation source such as a linear accelerator. High-energy photons are the most common type of radiation treatment in use today. Electron beams produced by a linear accelerator are used for tumors close to a body surface since they penetrate less into deeper tissues.

Protons are a newer form of treatment. Protons cause little damage to tissues they pass through but cause cell death in the cells at the end of the proton's path. In this way, proton beams may be able to deliver more radiation to the local area of the cancer while causing fewer side effects to normal tissues nearby. Although protons are used routinely for certain types of cancer, but are not yet used in other types of cancer. Some of the techniques used in proton treatment can also expose the patient to neutrons. Also, proton beam radiation therapy requires highly specialized equipment and is currently offered in only a few medical centers.

Neutrons are used for some cancers, such as cancers of the head, neck, or prostate. Neutrons can sometimes be helpful when other forms of radiation therapy do not work. Neutron use has declined over the years because of certain rather severe long-term side effects that neutrons cause.

There are several ways that different types of radiation can be delivered. One such delivery method is external beam radiation therapy or teletherapy. One type of radiation therapy commonly used involves photons. X-rays were the first form of photon radiation to be used to treat cancer. Depending on the amount of energy they possess, x-rays can be used to destroy cancer cells on the surface of an area, or penetrate to tissues deeper in the body. The higher the energy of the x-ray beam, the deeper the x-rays can go into the target tissue. Linear accelerators and betatrons produce x-rays of increasingly greater energy. Focusing radiation (such as x-rays) on a cancer site is called external beam radiotherapy. With modern radiation equipment, there is minimal scatter of x-ray energy outside the treatment beam. Scatter refers to the presence of radiation in the body outside the field of treatment. In radiation therapy, a sharply defined x-ray beam minimizes the side effects of treatment because only small amounts of radiation travel to other parts of the body.

There are certain known side effects associated with the currently available forms of radiotherapy. The radiation side effects experienced by the normal body tissues during and after radiotherapy can be loosely divided into acute effects and late effects. Acute radiation side effects constitute the acute reaction occurring during radiation and in the immediate weeks and months following treatment.

Radiation treatment is painless and without sensation, with the exception of some mechanical sounds produced by the treatment machine associated with the start and finish of the treatment. Some patients receiving radiation therapy will experience very little reaction, but in most patients, the normal tissues will develop some degree of radiation reaction. This reaction varies in amount and type, depending on the part of the body treated and the amount of normal tissue included in the radiation treatment.

Where large areas of a patient are treated, such as the whole abdomen or chest, the reaction experienced will be mainly of a general nature. When small areas are treated, the reaction will be confined to that area of the body that is radiated and to the individual tissues included in the treatment volume. In a small area treatment, any general reaction will be much less or absent altogether.

The side effects that patients may get from radiation therapy can cause pain or discomfort. When a cure is not possible, radiation may be used to shrink cancer tumors in order to reduce pressure. Radiation therapy used in this way can treat problems such as pain, bleeding or it can prevent problems such as blindness or loss of bowel and bladder control.

Some general side effect symptoms of radiotherapy include radiation nausea, hair loss, fatigue/malaise, and low blood count. The degree to which patients experience nausea following treatment is very variable. Some people will experience hardly any at all, whereas others will be troubled by nausea or vomiting during the early part of the treatment and, in some instances, throughout the treatment. If it occurs, nausea is likely to be worst from two to several hours after treatment. Hair loss will typically only occur within the radiation field. Scalp hair will typically only be affected if the head receives radiation. Some degree of tiredness and lack of energy is often experienced. A reduction in certain elements of the blood is often seen following radiation therapy. This reduction results from radiation exposure of bone marrow, and to a lesser extent, from direct damage to lymphocytes in the blood stream and lymph nodes. The patient's white cell count is often reduced, particularly the lymphocyte count, and the number of platelets is often reduced. The extent of reduction in white blood cells and platelets depends on the extent and intensity of the irradiation. These reductions are seldom enough to cause clinical problems, but if clinical problems do occur, an interruption in treatment for a few days is usually sufficient to allow recovery. Reduction in red cells does not typically occur to any degree in radiation treatment, but may occur from blood loss due to bleeding. Changes in the peripheral blood count are much more marked in patients who have also received chemotherapy.

When a small area of tissue is treated, organ specific side effects of often occur. Localized reactions can occur in any tissues exposed to radiation treatment.

Where the skin receives a significant dose of radiation, a reaction typically will develop. The reaction often progresses through erythema to dry desquamation and moist desquamation. The reaction may only progress part way through these steps. Healing occurs through the same steps in reverse. If desquamation has occurred, crusts will form which protect the re-epithelialisation occurring underneath and the crusts will only come away and not reform when the skin is healed underneath.

Each time radiation therapy is delivered, small amounts are absorbed by the skin over the area being treated. About 2 to 3 weeks after a patient's first radiation treatment, their skin may look red, irritated, sunburned, or tanned. Also, the skin may become dry or reddened from the therapy. Most skin reactions should go away a few weeks after treatment is finished. In some cases, though, the treated skin will remain darker than it was before.

Wherever mucous membranes are included in a radiation field similar reactions in those various mucous membranes often will be experienced: Whether in the mouth, pharynx, esophagus, trachea, bowel, bladder or rectum, mucositis may develop. As with the skin, the mucosa is reddened at first but then may be covered with a plaque-like fibrin similar to crusting of the skin. The mucous membrane remains moist and the surface covered by fibrin until the underlying mucosa is healed. Upon healing, the fibrinous plaque is lost.

The symptoms resulting from the inflammation, irritation, and dysfunction caused by the mucosal reaction depend on the site of the reaction. There may be discomfort, dysphagia, cough, hoarseness, tracheitis, dysuria, urinary frequency, diarrhoea and/or abdominal cramps. The management of these symptoms varies from mucosal site to mucosal site, but depends on the same principles as the care of skin reaction to radiotherapy.

Another type of tissue affected by small area radiotherapy is accessory glands. The acute effects of radiation typically will be felt by accessory glands producing saliva and mucous, for example. The reaction in these glands leads to a degree of stickiness, leading to oral discomfort, dryness, change in taste, irritating cough, and urinary or bowel symptoms, depending on the site of radiation. Another condition is called radiation pneumonitis, when the lung tissue becomes inflamed (swollen) and can occur within the first few months of treatment.

In contrast to the above acute side effects, the late effects of radiation treatment develop gradually over several months or years. The changes that result may be sufficiently slight as to cause no clinical symptoms, or so rare as to present minimal risk to the individual. Nevertheless, the late changes that do occur warrant notice and care in all patients who have received radiation treatment. In those few individuals with serious late effects (generally less than 5% of patients who have received high-dose radiation) the results are often disastrous and treatment is extremely difficult.

For example, radiation treatment can result in increased connective tissue fibrosis and scarring often associated with atrophy of accessory tissues. This fibrosis and scarring leads to some increased rigidity of tissues, less suppleness, and less resistance to injury.

In addition, the walls of small blood vessels may be thickened and distorted, leading to reduction in blood supply to some tissues. This particularly leads to less ability to deal with injury or trauma such as that resulting from infection or surgery.

Very rarely leukemia may result some five to twenty years after radiation exposure, due to bone marrow cells being damaged during radiation therapy. Similarly, cancer can result in a radiotherapy treatment area twenty or more years later than the treatment. However, the patient's risk of dying of the original disease, unless successfully treated, generally is much higher than the risk of developing cancer from the treatment. Nevertheless, the risk is there and is one of the reasons why benign diseases are not treated by radiation unless absolutely necessary.

In another example of late radiation effects, exposure of the gonads to radiation increases the risk of abnormal mutations and genetic changes. Most chromosome damage from radiation results in a failure of conception and not an abnormal child. Even if both parents have been exposed to radiation, the risks of abnormal children being produced are almost negligible.

In part because of concern over the side effects of radiotherapy, scientists have developed newer, more precise ways of giving external radiation therapy. These newer approaches allow the physician to focus the radiation more directly on the tumors. These newer forms of radiation do less damage to normal tissues, and allow the physician to use higher doses directed only at the tumors. Most of these methods are still fairly new, and their long-term effects are still being studied.

Newer machines allow the physician to conform the shape of the radiation beam to match the shape of the tumor. With conformal radiation, a special computer uses imaging scans (such as CT scans) to map the location of the cancer in the body in three dimensions. Radiation beams can then be directed to conform to the shape of the cancer. This helps to better protect the parts of the body in between the radiation beam and the cancer.

Three-dimensional conformal radiation therapy (3D-CRT) delivers shaped beams at the cancer from different directions. 3D-CRT uses special computers to precisely map the location of the tumor. Alternately or additionally, patients are fitted with a mold or cast to keep the body part still so the radiation can be aimed more accurately. By aiming the radiation more precisely, it may be possible to reduce radiation damage to normal tissues and better fight the cancer by increasing the radiation dose to the cancer.

Intensity modulated radiation therapy (IMRT) is a newer method similar to 3D-CRT. It conforms to the tumor shape like 3D-CRT, but also allows the strength of the beams to be changed to lessen damage to normal body tissues. This provides even more control in reducing the radiation reaching normal tissue while delivering a higher dose to the cancer. 3D-CRT may result in even fewer side effects.

A newer form of IMRT, known as helical tomotherapy, uses a linear accelerator inside a large “donut” that spirals around the body while the patient rests on a table during the treatment. Helical tomotherapy can deliver radiation from many different angles around the body. This may allow for even more precisely focused radiation.

Conformal proton beam radiation therapy is similar to 3D-CRT but it uses proton beams instead of x-rays. As previously discussed protons can only be put out by expensive equipment and requires expert staff. As of late 2007, fewer than half a dozen treatment centers in the United States offer it. Unlike x-rays, which release energy both before and after they hit their target, protons cause little damage to tissues they pass through and then release their energy after traveling a certain distance. This means that proton beam radiation may be able to deliver more radiation to the prostate and do less damage to nearby normal tissues. As with 3D-CRT and IMRT, early results are promising, but more studies will be needed to show a long-term advantage over standard external beam radiation.

Gamma rays are another form of photons used in radiotherapy. Gamma rays are produced spontaneously as certain elements (such as radium, uranium, and cobalt 60) release radiation as they decompose or decay. Each element decays at a specific rate and gives off energy in the form of gamma rays and other particles. X-rays and gamma rays generally have similar effects on cancer cells.

Another technique for delivering radiation to cancer cells is to place radioactive implants directly into a tumor or body cavity. This is called internal radiotherapy. Brachytherapy, interstitial irradiation, and intracavitary irradiation are types of internal radiotherapy. In internal radiotherapy, the radiation dose is concentrated in a small area. Internal radiotherapy is sometimes used for cancers of the tongue, uterus, prostate, and cervix. One of the advantages of this type of therapy is that there is less radiation exposure to other parts of the body.

The main types of internal radiation are 1) interstitial radiation, in which the radiation source is placed directly into or next to the tumor using small pellets, wires, tubes, or containers and 2) intracavitary radiation, in which a container of radioactive material is placed in a cavity of the body such as the vagina. X-rays, ultrasound, or CT scans are used to help the doctor put the radioactive source in the right place. The placement can be permanent or temporary.

Permanent (low dose rate) brachytherapy involves using small containers, called pellets or seeds, which are about the size of a grain of rice. They are placed directly into tumors using thin, hollow needles. Once in place, the pellets give off radiation for several weeks or months. Because they are so small and cause little discomfort, the pellets are simply left in place after their radioactive material is used up.

Temporary (high dose rate) brachytherapy involves temporarily placing hollow needles, tubes, or fluid-filled balloons into the area to be treated. Radioactive material can then be inserted for a short period of time and then removed. This process may be repeated over the course of a few days or weeks. Depending on how long the radioactive material is left in place, it may be necessary for the patient to stay in bed and lie fairly still to keep the implant from shifting.

For brachytherapy to be effective, the cancer typically must be no more than 2 inches in diameter and surgically accessible. Larger tumors may require surgery to reduce the size of the tumor before the radiation sources are implanted. Interstitial radiation is a local therapy. It is not commonly used for widely spread or multiple tumors. This type of therapy can be used for newly diagnosed or recurrent tumors, as a boost before or following standard external beam radiation therapy for newly diagnosed or recurrent cancers.

Interstitial radiation requires placement of catheters (tubes) into or near the cancer using CT or MRI-directed stereotactic surgical techniques. The sources of radiation, usually in pellet form, are then placed into the catheters. Depending on the isotopes used, the implant is removed either after a few days or several months, or left in place permanently. Steroids are commonly used with this therapy to decrease brain swelling. Different radioactive isotopes are currently being used as implants and others are being developed. Follow-up surgery to remove dead cancer cells is required in about 30% -40% of the patients receiving this therapy. Unlike external radiation, with interstitial radiation the patient is radioactive and precautions are needed until the implant is removed or until a predetermined amount of time has elapsed.

Several new approaches to radiation therapy are being evaluated to determine their effectiveness in treating cancer. For example, intraoperative radiation therapy (IORT) delivers radiation directly to the tumor or tumors during surgery. While the patient is under anesthesia, a surgeon locates the cancer. Normal tissues can be moved out of the way and protected during surgery, so IORT typically reduces the amount of tissue that is exposed to radiation.

Intra-operative radiotherapy is a technique for delivering radiation directly to the tumor at the time of the operation. A radiation boost delivered with high-energy electron beams can intensify the anti-tumor therapy in patients undergoing cancer surgery. Intra-operative radiotherapy can improve the precision of radiation, thus decreasing the damage to normal tissue.

A recent study was conducted involving 17 patients with primary or recurrent high-grade malignant gliomas, including glioblastoma multiform, who were treated after surgical resection with a single dose of intra-operative radiation therapy. For glioma patients, the 18-month survival rate was 56%. For patients with recurrent gliomas, the 18-month survival rate was 47% and the average survival time was 13 months. The researchers concluded that intra-operative radiation therapy is an attractive, tolerable and feasible treatment modality. Researchers will continue to evaluate what role, if any, intra-operative radiation therapy has for the treatment of glioblastoma multiforme.

Stereotactic radiosurgery is not really surgery but a type of radiation treatment that delivers a large, precise radiation dose to a small tumor area in a single session. It is most commonly used for brain tumors and other tumors inside the head. First, a head frame is attached to the skull to help precisely aim the radiation beams. Once the exact location of the tumor is known from the CT or MRI scans, radiation from a machine called a Gamma Knife can be focused at the tumor from hundreds of different angles for a short period of time.

Stereotactic radiation therapy is now a standard form of treatment for primary and metastatic brain cancer. The use of CT scans and MRI allows precisely focused, high-dose radiation beams to be delivered to a small brain cancer (usually 1 inch or less in diameter) in a single or multiple treatment sessions. The cancer can be located in an area of the brain or spinal cord that might be considered inoperable. Using special computer planning, this treatment minimizes the amount of radiation received by normal brain tissue. Because treatment is totally non-invasive, patients maintain their normal function throughout this process. Patients are completely awake and alert throughout the entire painless procedure. Stereotactic radiation therapy can be delivered as a single dose or in daily doses (fractionated) or more than one fraction per day (hyperfractionated).

Stereotactic radiation therapy is also used as a local “boost” following conventional radiation therapy, for a recurrent tumor when the patient has already received the maximum safe dose of conventional radiation therapy, as a substitute for surgery for a benign tumor (such as a pituitary, pineal region or acoustic tumor) or for a metastatic brain tumor.

Possible side effects of stereotactic radiation therapy include edema (swelling), occasional neurological problems and radiation necrosis (an accumulation of dead cells). A second surgery to remove the build-up of dead tumor cells may be required.

Two types of machines are used routinely to deliver stereotactic radiation therapy, Gamma Knife and Linac (adapted linear accelerators). The Gamma Knife contains 201 radioactive cobalt sources, which can all be computer-focused onto a single area. The patient is placed on a couch and then a large helmet is attached to the head frame. Holes in the helmet allow the beams to match the calculated shape of the cancer. The couch is then pushed into a globe that contains radioactive cobalt. The most frequent use of the Gamma Knife has been for small, benign tumors, particularly acoustic neuromas, meningiomas and pituitary tumors. For larger tumors, partial surgical removal might be required first. The Gamma Knife is also used to treat solitary metastases and small malignant tumors with well-defined borders.

In another form of cancer radiotherapy, an adapted linear accelerator delivers a single, high-energy beam that is computer-matched to the cancer. The patient is positioned on a sliding bed around which the linear accelerator circles. The linear accelerator directs arcs of radioactive photon beams at the tumor. The pattern of the arc is computer-matched to the tumor's shape. This reduces the dose delivered to surrounding normal tissue. A similar approach uses a movable linear accelerator that is controlled by a computer. Instead of delivering many beams at once, the machine moves around to deliver radiation to the tumor from different angles. Several machines do stereotactic radiosurgery in this way, with names such as X-Knife, CyberKnife, and Clinac. Another technique uses particle beams of protons or helium ions to deliver the radiation to the tumor in this way.

Stereotactic radiosurgery typically uses a single session to deliver the whole radiation dose, though it may be repeated if needed. Sometimes doctors give the radiation in several treatments to deliver the same or slightly higher dose (fractionation). This is sometimes called fractionated radiosurgery or stereotactic radiotherapy. Clinical trials are under way to study how well stereotactic radiosurgery and stereotactic radiotherapy work alone and when used with other types of radiation therapy.

Particle beam radiation therapy differs from photon radiotherapy in that it involves the use of fast-moving subatomic particles to treat localized cancers. A very sophisticated machine is needed to produce and accelerate the particles required for this procedure. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation.

Two types of investigational drugs are being studied for their effect on cells undergoing radiation. Called radiosensitizers, these drugs make the tumor cells more likely to be damaged by radiation. Other drugs, called radioprotectors, protect normal tissues from the effects of radiation. Hyperthermia, or the use of heat, is also being studied for its effectiveness in sensitizing tissues to radiation.

Known methods of radiotherapy present certain challenges, including unwanted side effects, prohibitive cost, and specialized facilities. The above challenges, and others not described, may be addressed in part by certain embodiments of the present invention. Other features and advantages of the present invention will be apparent from the following detailed description.

### BRIEF SUMMARY OF THE INVENTION

Certain embodiments of the present invention include a composition for the treatment of disease comprising a microparticle having an outer surface, a targeting agent linked to the outer surface of the microparticle, and at least one alpha emitting radionuclide carried by the microparticle. In some embodiments, one alpha emitting radionuclide is contained at least partially within the microparticle. In some embodiments, at least one alpha emitting radionuclide is linked to the outer surface of the microparticle. In some embodiments, an echogenic gas is within the microparticle. In some embodiments, the targeting agent is an antibody. In some embodiments, the antibody is a tumor recognizing antibody. In some embodiments, a therapeutic agent is carried by the microparticle. In some embodiments, the therapeutic agent is a cancer chemotherapeutic agent.

Certain embodiments of the present invention include a method for the treatment of disease comprising delivering a microparticle to a treatment site of a patient, the microparticle having a targeting agent linked to an outer surface of the microparticle and the microparticle carrying at least one alpha radiation emitting radionuclide. In some embodiments, the method includes applying ultrasound energy to the treatment site. In some embodiments, the method includes determining the location of the microparticle using an imaging modality matched to an imaging marker carried by the microparticle. In some embodiments, the disease is cancer, vulnerable plaque, or chronic synovitis.

Certain embodiments of the present invention include a method for the local treatment of a disease in a patient comprising delivering a composition of microparticles to the patient, the microparticles having a targeting agent linked to an outer surface of the microparticle and the microparticle carrying at least one alpha radiation emitting radionuclide and an imaging marker. In some embodiments, the method includes locating microparticles near a local treatment site of a patient using an imaging modality. In some embodiments, the method includes applying ultrasound energy to the local treatment site when the microparticles are located near the local treatment site.

### BRIEF DESCRIPTION OF SEVERAL VIEWS OF THE DRAWINGS

FIG. 1 illustrates a targeted microbubble for radiotherapy in accordance with certain embodiments of the present invention.

### DETAILED DESCRIPTION OF THE INVENTION

Embodiments of the invention relate to imaging and therapeutic agents, which are comprised of a molecular targeting entity, a diagnostic or therapeutic entity, and a linking carrier. In certain embodiments, the carrier is a microbubble. Certain methods of the invention include incorporating and/or labeling microbubbles of various composition with radionuclides for therapeutic purposes singly and/or in combination with radionuclide, magnetic resonance (MR), positron emission tomography (PET), single photon emission computer tomography (SPECT) markers for imaging, and/or therapeutic agents such as drugs for combination therapy and radiosensitizers, and/or cancer-specific antibodies or other molecules, which will be delivered to the targeted cell, tissue or organ of interest intravenously or intrarterially, and either interacted passively with the tissue or organ by presentation through the blood stream or delivered actively intracellularly by rupturing the microbubbles with specified frequencies of ultrasound to either penetrate the cell membrane or open up channels in the membrane that allows the radionuclide, drug or other agent to be delivered into cells. Certain methods of the invention include incorporating prodrugs, medications, plasmids and gene encoding proteins, antibodies and other molecules into or on the surface of microbubbles. Certain embodiments and/or methods of the invention use ultrasonic energy, which may be delivered locally, to part of the body, or to the whole body. Standard echocardiographic equipment, whole body ultrasound, and/or high intensity focused ultrasound (HIFU) can be employed in certain methods and/or embodiments of the invention.

Certain embodiments and/or methods of the present invention provide some or all of the following benefits: local dose control of radiotherapy; cellular penetration of a radionuclide; radionuclide delivery targeted by ligands and/or targeted by activation; and/or a combination of imaging and radiotherapy.

As used herein, the term “targeted” and its variations refer generally to the method of selectively addressing a specific site in the body. This selective addressing can be accomplished through the use of molecules that recognize or have an affinity for specific sites, such as receptor-ligand pairs or antigen-antibody pairs. Selective addressing can also be accomplished by application of external energy to a specific site in the body, such as the application of ultrasound to activate a microbubble and cause it to deliver a therapeutic payload. Further, a combination of “molecular targeting” and “activation targeting” may be used. Thus, as used herein, the term “targeted” and its variations embrace each of the these meanings as is appropriate from the context.

New approaches in the treatment of cancer are necessary to overcome the limited therapeutic efficacy of currently available therapeutics. Conventional therapies often have negative side effects which severely limit the therapeutic doses that can be administered thus severely compromising efficacy of the treatment and affecting the patient's overall health and quality of life. As a result, the disease often recurs in time due to the surviving and spreading of cancerous cells from the original tumor to other areas in the body.

The use of alpha emitters as the therapeutic radionuclides in certain embodiments of the present invention presents certain advantages over other radionuclides. Different radionuclides have been shown to exhibit properties suitable for treating tumors. For example, radionuclide therapy using chromic phosphate (P-32), which is a low-LET (linear energy transfer) beta-emitter, has exhibited some level of success. A five-year survival rate of 81 percent for the treatment of microscopic disease has been reported for patients with stage I and stage II disease. Young et al., N. Eng. J. Med., 322:1021 (1990). However, P-32 is sparsely ionizing and its effectiveness is dependent on cellular oxygen. In contrast, some of the advantages of alpha emitters are explained below.

Alpha-emitting radionuclides have been found to be effective in the treatment and eradication of microscopic carcinoma in animal models. This is believed to be a result of the densely ionizing radiation that is emitted during alpha-decay, and the cellular oxygen independence of the effect of an alpha particle on the disease.

It has been shown that lead-212 (Pb-212) and astatine-211 (At-211) are effective in the treatment and eradication of microscopic carcinoma. The effectiveness of Pb-212 in treating the carcinoma is due to its subsequent decay to Bi-212, which is an alpha-emitting radionuclide. Pb-212, itself, is not as effective as the alpha-emitting Bi-212 radionuclide.

Known processes for producing alpha particle-emitting nuclides such as At-21 1 are limited in that they generally require the use of particle accelerators for production of the nuclides. Moreover, the radionuclides so produced are often contaminated with radio-impurities that are difficult to filter out or otherwise remove from a desired nuclide. It has also been found that such nuclides that are administered intraperitoneally using a complexing agent such as Pb-212/ferrous hydroxide do not have the desired property of even distribution.

Bismuth-212, which, as noted above, is an alpha-emitting radionuclide, has recently been found to exhibit the desirable properties associated with At-211 in providing highly ionizing radiation and exhibiting cellular oxygen independence. Moreover, certain formulations of Bi-212 have also been found to overcome the distributional problems encountered with complexed Pb-212 and At-211 upon intraperitoneal administration. In addition, Bi-212 has a half-life of 60.6 minutes, which makes this isotope useful for intraperitoneal treatment because it emits its radiation while its distribution in the peritoneal fluid is uniform.

U.S. Pat. No. 6,126,909, to Rotmensch, et al. provides further details regarding alpha emitters, and Bismuth-212 in particular, and is incorporated by reference in its entirety into the present disclosure.

Alpha-emitting radionuclides have physical properties that make them attractive for therapy. Unlike X-rays and gamma-rays, alpha-emitters have a very high linear energy transfer (LET). For alpha particles for effectiveness is due to the amount of energy deposited per unit distance traveled or LET. For alpha particles, the LET is approximately 400 times greater than that of beta-particles (80 keV/μm vs. 0.2 keV/μm). In human tissue, all of an alpha-emitter's energy is typically deposited in the first few microns of travel, resulting in a very high local radiation dose. With alpha-emitters it is preferable that the distribution of the radionuclide in the target tissue be uniform, because the range in matter is so short. Beta-emitters are more forgiving because the beta-particles travel 5-10 mm through tissue and, therefore, typically deliver a dose to the entire target organ even if their distribution is less than ideal. Because these radionuclides are used to destroy cells, one must be very sure that localization of these nuclides in target tissue is optimal. This means that the target-to-nontarget ratio of activity should be very high (>25:1). If the radionuclide purity is not very high (>95%), then contaminating radionuclides can significantly increase the radiation dose to the target and surrounding tissues and, possibly, to areas of the body remote from the site of interest. If the radiochemical purity is not high, then the radioisotope is in the wrong radiochemical form. In this case, it might localize in an undesirable place (e.g., bone marrow) instead of in the desired target organ. The potential for a resulting catastrophic illness (leukemia and aplastic anemia) resulting from this poor biological distribution is quite significant. Thus, one must perform the appropriate quality control procedures to ensure suitability of drug administration to humans. This will decrease the risk of undesirable effects on the patient.

A number of factors must be considered in selecting an alpha-emitting radionuclide for therapeutic applications. With regard to its nuclear properties, the fraction of decays involving the emission of alpha-particles should be high and, for many applications, the absence of beta-particles also would be advantageous. In addition, the emission of gamma-rays or x-rays with an energy appropriate for external imaging would be helpful for monitoring in vivo distribution. Finally, the physical half-life of the radionuclide should be long enough to permit convenient radiosynthesis. Other considerations in radionuclide selection are dependent on the nature of the intended radiotherapeutic approach. Radiochemical strategies must be available to label the carrier molecule in reasonable yield and in such a way that the labeled molecule has adequate stability in vivo, or alternatively, that the labeled catabolites are excreted rapidly. In addition, the half-life of the radionuclide should be compatible with the dynamics of tumor localization and retention of the intended carrier molecule.

Numerous radionuclides have been identified which de-excite by the emission of alpha-particles. However, the vast majority lack the characteristics noted above, possessing either too long a half-life or too complex a decay scheme to merit serious consideration for radiotherapeutic applications. Others may have acceptable nuclear decay properties but cannot be produced in sufficient quantity and isotopic purity to permit clinical use. As a result of these requirements, the only alpha-emitting radionuclides which have received serious attention for endoradiotherapy are Bi-212 and At-211.

For applications well matched to their short range in tissue, alpha-particles offer a number of advantages for radiotherapy from a radiobiological perspective. As a result of their short range and high energy, alpha-particles are radiation of high linear energy transfer (LET). The LET for alpha-particles increases with decreasing particle energy as they pass through matter. The LETₘₑₐₙ for the alpha-particles of At-211 is about 100 keV μm-1, a value which is close to that at which the relative biological effectiveness of ionizing radiation is highest. This is due to the fact that the average separation between ionizing events at this ionization density nearly coincides with the diameter of the DNA double helix, increasing the probability of double-strand breaks. In comparison, Y-90 beta-particles have a LETₘₑₐₙ of only 0.2 keV μm-1.

Because of the higher probability for creating double-DNA-strand breaks, which are generally not repairable, the cytotoxic effectiveness of alpha-particles is much less dependent on dose rate than is that of beta-particles. This is an advantage since, in many cases, the dose rates achieved with targeted radiotherapy have not been high. Another advantage of high-LET radiation is that it is associated with a low oxygen enhancement ratio so that it is possible to treat both oxic and hypoxic cell populations. Finally, the cytotoxicity of high-LET radiation is nearly independent of cell cycle position. Thus, a strong radiobiological rationale exists for the use of alpha-particles in targeted radiotherapy.

Tumor size and geometry are important factors governing the selection of the type of radiation for a particular therapeutic application. The proximity of the targets cells to highly radiation-sensitive normal tissues also should be considered. As the tumor size decreases, the potential advantage of At-211 alpha-particles compared with beta-particles should increase, even when differences in relative biological effectiveness are not taken into account.

This can be illustrated by comparing the properties of At-211 alpha-particles with those of the beta-particles emitted by Y-90. Although the Y-90 beta-particles have a maximum energy of 2.28 MeV, about one third that of At-211 alpha-particles, their mean and maximum ranges in tissue are about 4 and 11 mm, respectively, compared with 55-80 μm for At-211 alpha-particles. The consequences of this difference can be appreciated by calculating the At-211 alpha-particle: Y-90 beta-particle absorbed fraction ratio and observing its variation with tumor size. Values of 9:1 and 33:1 for this parameter have been calculated for 1 and 0.2 mm tumors, respectively. Under single-cell conditions, about 1000 times more cell-surface decays of Y-90 would be required to achieve the same cell killing as At-211. Most strategies for applying Bi-212 and At-211 labelled radiopharmaceuticals have attempted to capitalize on the short range of their alpha-particles in tissue. Micrometastatic disease as well as tumors characterized by free-floating cells in the circulation, such as lymphomas, might be amenable to treatment with targeted alpha-particle radiotherapy. Another type of application which has received considerable attention is the treatment of cancers that spread as thin sheets on the surface of body cavities, such as neoplastic meningitis and ovarian cancer. Intracavitary disease is a particularly appropriate setting since administration of the agent directly into the body space hastens the delivery of these relatively short-half-life radionuclides while reducing the exposure of normal tissue to them.

The development of methods for calculating radiation dosimetry of alpha-emitting radiopharmaceuticals is useful for at least two reasons. First, such information could facilitate the evaluation of tumor and normal cytotoxicity data obtained in preclinical models. Secondly, if clinical trials with alpha-emitting therapeutic agents are initiated, it will be useful to attempt to relate tumor and normal tissue effects to some parameter associated with the dose of radiation absorbed.

Conventional calculations of the absorbed dose of radiation such as those of the Medical Information Radiation Dose (MIRD) committee consider radionuclide activity to be uniformly distributed in source organs. However, because the range of alpha-particles in tissue is only a few cell diameters, it is unlikely that the tracer distribution in these volumetric dimensions will be homogeneous. Differences in the blood flow, permeability, tumor interstitial pressure and cellular concentration of the molecular target (for example, antigen or receptor) can all contribute to a heterogeneous tracer distribution. Furthermore, the stochastic nature of radiation will lead to a distribution of energy deposition among the radiosensitive targets, such as the cell nuclei.

Targeted radiotherapy with alpha-particles typically uses a microdosimetric perspective. Results are generally expressed as the specific energy, defined as the ratio of the energy deposited to the mass of the target. This is a stochastic parameter, with the mean specific energy equivalent to the dose absorbed. Two general approaches have been used: Monte Carlo calculations and analytical microdosimetry using Fourier transform techniques.

The ability to monitor the time-dependent distribution of targeted radiotherapeutic agents both in tumors and in normal tissue by external imaging can provide useful information for optimizing treatment strategies. In addition, such information can be used to determine the suitability of a given agent for a particular patient. The tissue distribution of the imaging radionuclide mimic that which will occur when the therapeutic radionuclide is used for labeling. Both I-123 and I-124 are attractive for use with At-211 from an imaging perspective and iodine is chemically similar to astatine. Unfortunately, the tissue distribution of radio-iodinated compounds rarely reflects that of their At-211 labeled analogues, so alternative approaches, such as those according to certain embodiments and methods of the present invention, are needed.

It may be possible to image the polonium K x-rays emitted during the electron-capture decay of At-211. A confounding factor is emission of low abundance but high-energy gamma-rays (570, 688 and 898 keV) by At-211 which can degrade image quality. The ability to image At-211 distributions was studied using a variety of single-photon emission tomographic (SPECT) imaging methods. Penetration fractions with medium-energy, low-energy high-resolution and low-energy super-high-resolution collimators were 7, 22 and 41%, respectively. The ability to quantify At-211 distributions in simple phantom geometries was demonstrated.

Low-dose rate alpha-radioimmunotherapy seems to be beneficial against macroscopic tumors as well as single tumor cells. There may be both advantages and disadvantages of using low dose rates. Disadvantages may include tumor tissue repair due to proliferation and possible DNA repair, although the latter is less likely since alpha radiation causes mainly irreparable double-strand breaks in the DNA. The therapeutic level of Th-227 found to be effective in this study was quite modest. The amount of Ra-223 generated would probably not limit the use of Th-227, as indicated by the modest toxicity shown in recent clinical data on Ra-223 in patients with prostate and breast cancer.

The beta-emitting, commercially available RIC Y-90 tiuxetan-ibritumomab, which also targets CD20 presenting cells, had significantly less effect than Th-227 DOTA-p-benzyl-rituximab. The uptake of I-125 ibritumomab-tiuxetan in tumors was significantly lower than the uptake of Th-227 DOTA-p-benzyl-rituximab. The immunoreactivity of I-125 ibritumomab-tiuxetan was 57%, which is acceptable. The tumor uptake in percentage of injected dose per gram 7 days after injection was 26% for Th-227 DOTA-p-benzyl-rituximab, 3% for I-125 ibritumomab-tiuxetan, and 19% for 1-125 rituximab. Thus, labeling of rituximab with I-125 did not alter the tumor uptake significantly, indicating that Y-90 tiuxetan-ibritumomab is not as suitable for therapy of mice with lymphoma xenografts as radiolabeled rituximab. Consistently, single injections of 278 to 370 MBq/kg Y-90 tiuxetan-ibritumomab had to be administered to achieve a significant increase in median survival time in a Ramos xenograft model. The standard patient dosage of Y-90tiuxetan-ibritumomab is 15 MBq/kg. It is noteworthy that Th-227 rituximab was significantly more effective than the clinically proven Y-90tiuexetan-ibritumomab.

The recently developed method yielding stable constructs of Th-227 DOTA-p-benzyl-IgG in therapeutic quantities, and the demonstration of safe, efficacious use against a macroscopic tumor model, using modest dosages of isotope, suggest that clinical use of such targeted drugs is feasible. The 18.72-day half-life of Th-227 would allow the drugs to be manufactured at a central radiopharmacy and shipped throughout the world. Because of the extraordinary potency of the alpha-emitting Th-227 radionuclide, a limited amount of radioactivity would be required for therapeutic human use, permitting an economic and safe outpatient use. In addition, the half-life of Th-227 may allow time to maximize the uptake in macroscopic tumors.

Although the mechanisms by which radiation induces cell death are not completely understood, several processes have been implicated. Radiation induces single- and double-stranded DNA breaks, causes apoptosis, and initiates overexpression of p53, leading to delays in the G₁ phase of the cell cycle. Death of cells exposed to alpha-particles occurs only when the particles traverse the nucleus; high concentrations of alpha-particles directed at the cytoplasm have no effect on cell proliferation.

Linear energy transfer (LET) and relative biologic effectiveness (RBE) are essential radiobiologic concepts. LET refers to the number of ionizations caused by that radiation per unit of distance traveled. alpha-particles have a high LET (approximately 100 keV/μm), whereas, beta-particles have a far lower LET (0.2 keV/μm). The RBE for a type of radiation refers to the dose of a reference radiation, usually x-rays, that produces the same biologic effect as the type of radiation in question. The RBE of a type of radiation is in part related to its LET. The RBE of alpha-particles for cell sterilization ranges from 3 to 7, depending on emission characteristics.

The dependency of RBE on LET can be explained by several differences in the type and extent of cellular damage caused by low- and high-LET radiations. First, high-LET radiation generally causes more irreparable clustered and double-stranded DNA breaks than low-LET radiation. The maximum rate of double-stranded DNA breaks occurs at LETs of 100-200 keV/μm, since the distance between ionizations caused by the radiation at these LETs approximates the diameter of double-stranded DNA (2 nm). Second, high-LET radiation causes more severe chromosomal damage, including shattered chromosomes at mitosis and complex chromosomal rearrangements, than low-LET radiation. Third, high-LET alpha-irradiation causes more pronounced G₂-phase delays than low-LET gamma-irradiation. The mechanisms behind these differences in cell cycle effects have not been fully elucidated but may be related to differences in gene expression induced by low- and high-LET radiations.

The different physical properties of alpha- and beta-particles confer theoretic advantages and disadvantages to each, depending on the clinical situation. Since the range of beta-emissions extends for several millimeters, therapy with isotopes such as I-131, Y-90, and Re-188 can create a “crossfire effect,” destroying tumor cells to which the radioimmunoconjugate is not directly bound. In this way, beta-emitters can potentially overcome resistance due to antigen-negative tumor cells. Conversely, longer-range beta-emissions may also produce nonspecific cytotoxic effects by destroying surrounding normal cells. These characteristics make beta therapy better suited for bulky tumors or large-volume disease.

In contrast, alpha-particles may be better suited to the treatment of microscopic or small-volume disease since their short range and high energies potentially offer more efficient and specific killing of tumor cells. In a microdosimetric model using single-cell conditions, 1 cell-surface decay of the alpha-emitter At-211 resulted in the same degree of cell killing as approximately 1,000 cell-surface decays of the beta-emitter Y-90. Based on these considerations, alpha-particle therapy has been investigated in a variety of settings, including leukemias, lymphomas, gliomas, melanoma, and peritoneal carcinomatosis.

Alpha emissions have high energies of several MeV, exhibit very short path lengths (<80 μm), and are associated with a high probability of producing cytocidal DNA double-strand breaks. An individual cancer cell can be killed by interaction with only a few and possibly with only a single alpha particle. Moreover, the path length of alpha particles is short enough to avoid damaging nontargeted regions. Homogeneous antibody distribution within a tumor is, however, useful if a bystander effect is to be observed on antigen-negative cells. A lack of homogeneous targeting may be more significant for solid tumors, which are often poorly vascularized and have high interstitial pressure, due to poor lymphatic drainage. Consequently, alpha emitters may be most effective in internal radiation therapy of radio-immunotherapy (RIT) directed against blood-borne tumor cells, micrometastatic disease, and cancer cells near the surface of cavities. Cancers that are greater than 1 to 2 mm in size have an independent blood supply and are vascular, and many metastases are blood borne, and so are located near a blood vessel. This includes the most commonly encountered cancers such as breast, prostate, malignant melanoma and essentially all solid tumors. Bismuth-213, Pb-211, and Pb-213 are attractive alpha-emitting radionuclides that are now available for clinical use. The Pb-212 precursor with a longer half-life can also be used to generate Bi-212 in vivo. Another promising alpha-emitter, with a longer half-life of 7.2 hours, is At-211.

Most brachytherapy and radio-immunotherapy (RIT) uses beta decay. The disadvantages with beta emission are that a neutron breaks down, changing to a proton and emitting a high-energy electron (beta particle) and raising the atomic number by one without changing the mass number. Given the length of their path, beta emissions are appropriate for treating tumors larger than 0.5 cm. In addition, not every cell needs to be targeted with a radionuclide conjugate. Bombardment of adjacent tumor cells by multiple beta particles results in enhanced killing through cross-fire, partially compensating for a lack of homogeneity of antigen expression from cell to cell. In theory, one might choose among beta emitters based on the size of the tumor. Shorter-range beta emitters such as I-131 and Cu-67 might be used to treat micrometastatic disease, where a greater fraction of their decay energy would be deposited within small tumor cell clusters. Conversely, more energetic, longer-range beta emitters such as Y-90 could destroy larger tumor deposits and eliminate tumor cells that had escaped direct targeting due to lack of antigen expression or poor vascularity.

Beta emission from radioisotopes kills tumor cells but also kills normal cells. As blood circulates through the bone marrow, beta decay from circulating radionuclide conjugates irradiates bone marrow cells producing myelosuppression. Sites of specific binding of radionuclide conjugates can also impact on myelotoxicity. In trials of RIT for lymphoma patients, greater radiation doses were delivered to bone marrow involved with lymphoma than to bone marrow that was lymphoma free.

I-131 was the first isotope used in radiotherapy, but it is not optimal for RIT of larger tumor deposits. I-131 produces low energy beta particles, emits unwanted beta radiation, and exhibits a short biological half-life because of the action of tissue dehalogenases. Myelosuppression can follow I-131 antibody treatment because of the radiation dose that the bone marrow receives from circulating conjugates. Y-90 emits only beta particles of appropriate energy for therapy but still exerts myelosuppression. The extent of heterogeneity of dose deposition in tumor is highly dependent on the antibody characteristics and radionuclide properties and can enhance therapeutic efficacy through the selective dose delivery to the radiosensitive areas of tumor. Radionuclide characteristics can affect the heterogeneity of dose deposition within viable and necrotic areas of a tumor. When I-131 and Y-90 labeled radioconjugates were compared directly, I-131 generally delivered a higher dose throughout the tumor, even though the instantaneous dose-rate distribution for 90Y was more uniform.

The use of monoclonal antibodies to deliver radioisotopes directly to tumor cells has become a promising strategy to enhance the antitumor effects of native antibodies. Since the alpha- and beta-particles emitted during the decay of radioisotopes differ in significant ways, proper selection of isotope and antibody combinations is important to making radioimmunotherapy a standard therapeutic modality. Because of the short pathlength (50-80 μm) and high linear energy transfer (˜100 keV/μm) of alpha-emitting radioisotopes, targeted alpha-particle therapy offers the potential for more specific tumor cell killing with less damage to surrounding normal tissues than beta-emitters. These properties make targeted alpha-particle therapy ideal for the elimination of minimal residual or micrometastatic disease. Radioimmunotherapy using α-emitters such as Bi-213, At-211, and Ac-225 has shown activity in several in vitro and in vivo experimental models. Clinical trials have demonstrated the safety, feasibility, and activity of targeted alpha-particle therapy in the treatment of small-volume and cytoreduced disease.

Other recent radiotherapy research has focused on the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the body's immune system to produce tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. The benefit to this approach is that it can reduce the risk of radiation damage to the body's healthy cells. This technique depends upon both the identification of appropriate radioactive substances and determination of the safe and effective dose of radiation that can be delivered in this way.

A significant benefit of the antibody approach is that monoclonal antibodies generally only target cancer cells, sparing healthy cells from destruction. This is in contrast to chemotherapy or radiation, which do not differentiate between cancer cells and healthy cells in the body, leading to potentially destructive side effects.

Researchers have conducted an early phase clinical trial involving the surgical removal of the cancer followed by an injection of a radioactive isotope linked to a monoclonal antibody called Iodine-131 Antitenascin 81C6 (I-labeled 81C6). Antitenascin 81C6 identifies cancerous glioma cells by recognizing small proteins displayed on the surface of the cancer cells, called tenascin. When antitenascin binds to the cancerous glioma cells, the immune system is stimulated to attack the cancer cells. I-131 is a radioactive isotope substance that is attached to antitenascin 81C6. Radioactive isotopes kill cancer cells by spontaneously emitting forms of radiation. When antitenascin binds to cancer cells, the attached I-131 destroys these cells by emission of its radiation. I-labeled 81C6 not only provides two separate treatment strategies, but also allows the delivery of greater amounts of radiation directly to the cancer cells, while minimizing radiation exposure to normal cells. In this study, I-labeled 81C6 was injected directly into the cavity of the brain from which the cancer was removed in 42 patients with malignant gliomas who had not received prior treatment. The average duration of survival for patients was extended over standard treatment to one and half years. Some patients experienced neurological complications from the procedure, including seizures, memory loss, an inability to coordinate muscle movement and slight weakness on one side of their body.

The integrity of a radioimmunoconjugates can be susceptible to catabolism after internalization into a target cell or to the direct effects of radioactive decay. Therefore, in vivo stability of a radioconjugate is required to maximize delivery of isotope to tumor and to prevent toxicity. A variety of methods are used to conjugate radioisotopes to antibodies, depending primarily on the nature of the radioisotope.

At-211 is a halogen, like I-131, and is usually labeled directly to antibodies by incorporation of an aryl carbon-astatine bond into the antibody. Methods used to create the aryl carbon-astatine bond usually involve an astatodemetallation reaction using a tin, silicon, or mercury precursor. Other radioisotopes require bifunctional chelators for linkage to antibodies. Chelators derived from DTPA include the cyclic dianhydride derivative and the cyclohexylbenzyl derivative (CHX-A-DTPA). CHX-A-DTPA is effective at chelating bismuth to antibodies, resulting in stable constructs that have been used effectively in clinical trials. The macrocyclic ligand 1,4,7,10-tetraazacyclododecane tetraacetic acid (DOTA) and its derivatives have been used effectively for labeling of antibodies with Ac-225. A 2-step procedure was developed in which Ac-225 is first conjugated to DOTA-SCN followed by labeling of this construct to antibody.

The Bi-213 labeled humanized anti-CD33 monoclonal antibody, HuM195, was translated to a landmark clinical trial at Memorial Sloan-Kettering Cancer Center. Eighteen patients with advanced myeloid leukemia were treated in a Phase I dose-escalation trial and with myelosuppression in all patients along with transient minor liver function abnormalities. Doses of up to 37 MBq kg-1 (1 mCi kg-1) were safely administered. Uptake of Bi-213 was demonstrated by c-camera imaging to be in the bone marrow, liver, and spleen, without significant uptake in other organs, and most importantly, absent from the kidney. Absorbed dose ratios between marrow, liver, and spleen and the whole body were 1000 times greater with Bi-213 HuM195 than with previously evaluated HuM195 radiolabeled with beta-emitters. Fourteen out of eighteen patients had a reduction in the percentage of bone marrowblasts after therapy. There were no complete remissions thereby demonstrating the difficulty of targeting an adequate number of Bi-213 atoms to each leukemic blast at the specific activities used in this trial. A Phase I/II study followed wherein patients were first treated with chemotherapy to achieve partial cytoreduction of the leukemic burden followed by Bi-213 HuM195. Greater than 20 patients with acute myeloid leukemia were treated with cytarabine (200 mg −2 d-1 for 5 d) followed by Bi-213 HuM195 at 4 dose levels (18.546.25 MBq kg-1 [0.5-1.25 mCi kg-1]). Prolonged myelosuppression was dose limiting at the highest dose level. Complete responses, complete responses with incomplete platelet recovery, and partial responses were achieved at the two highest dose levels. These preliminary results indicate that sequential administration of cytarabine and Bi-213 HuM195 can lead to complete remissions in patients with acute myeloid leukemia. These studies have recently been extended to a Phase I study using Ac-225.

Peptides, as opposed to monoclonal antibody targeted alpha-therapy, have also been recently investigated to take advantage of both rapid targeting with cellular internalization combined with rapid clearance pharmacokinetics. A melanoma-targeting peptide, (DOTA)-Re(Arg11)CCMSH, was radiolabeledwith Pb-212 for biodistribution and therapy studies carried out in a B16/F1 melanoma-bearing murine tumor model. Treatment with 1.85, 3.7 and 7.4 MBq (50, 100, and 200 ICi) of Pb-212[DOTA]-Re(Arg11)CCMSH extended mean survival to 22, 28, and 49.8 days, respectively, as compared with a 14.6-day mean survival of the controls; 45% that received 7.4 MBq (200 ICi) surviving disease-free.

The somatostatin analogue [DOTA0, Tyr3]octreotide (DOTATOC) was labeled with Bi-213. Significant decreases in tumor growth rate were observed in rats treated with >11 MBq(300 ICi) of 213Bi-DOTATOC 10 days post-inoculation with tumor compared with controls (P<0.025). Treatment with >20 MBq (540 ICi) resulted in greater tumor reduction.

Cancer cells originate at one site and spread through the body at different rates. Current therapy relies upon surgical intervention to remove macroscopic tumors and irradiation of the tumor site with gamma rays to treat the remaining microscopic tumors. Chemotherapy is used to attack any residual or non-resectable disease, either at the surgical site or elsewhere in the body. Unfortunately, such measures rarely eradicate all of the residual disease. Complementary and/or alternate therapies are needed to eradicate the remaining tumor cells. Radioimmunotherapy targets therapeutic radiation to cancer cells anywhere in the body through the use of monoclonal antibodies. These targeting moieties identify and deliver the radiation to the tumor cells without causing significant damage to normal tissues. While monoclonal antibodies are made to selectively bind onto specific target molecules, they often lack the necessary therapeutic efficacy and the ability to offer a significant advantage over conventional therapies. Efforts to achieve greater therapeutic effects on the basis of antibody constructs, which include conjugates with chemotherapeutic compounds or Beta particles emitting isotopes, have provided encouraging results, but point to the need for a more focused modality for selective cell-kill.

Full realization of the monoclonal antibodies' inherent benefits could be achieved by combining their specific targeting characteristics with the potency and target range of the alpha particle emitting isotopes, bismuth-213, actinium-225 and Pb-211. These isotopes provide for the required selectivity and potency to directly kill its target cells without any dependency on the patient's immune system or need for a biological conversion into an active compound.

The key to alpha particle therapy is the control of the power of the alpha particles, which translates to an enhanced ability to kill tumor cells, while reducing the potential or severity of side effects. Alpha particles release more energy over a much shorter distance than beta irradiation, currently employed in radio-immunotherapeutic approaches. In addition, the isotopes chosen have a short half-life, limiting the presence of radiation in the body after they have executed their therapeutic effect. Use of alpha-particles as cancer killing agents instead of beta particles is more attractive for a combination of reasons: (1) The alpha's energy is 30 x greater than that of a beta (typically 6 MeV versus 200 keV); (2) The electric charge is double (+2 versus −1); (3) The mass is 7,000 x heavier (4 mass units versus 1/1800). As a result, the effective range of alpha particles in tissue is about 5 cell diameters compared with hundreds or thousands of cell diameters for beta particles.

The amount of energy dissipated per unit track length of an alpha particle is 1000× greater than that for a beta particle. Non-elastic collisions cause three times as much cell killing per unit of energy dissipated in tissue, proportionately increasing the effectiveness of cell killing. Because the effective range for alpha particles is less than 5 cell diameters, the killing is typically confined to tumor cells and thus collateral damage to normal tissue is minimized. The short penetration range and the short half-life of the therapeutic alpha particle emitting isotopes lead to no significant effect on normal tissues and no residual buildup of radiation in the body resulting in a far greater overall health benefit and improved quality of life. In clinical trials, there were no serious effects on any tissue or organ other than target tissue.

There are three principal contributing components, which are coordinated and managed for development and commercialization of alpha particle immunotherapy technology. These include monoclonal antibodies, chelators, and radioisotopes.

Monoclonal antibodies are used to target the alpha particle therapy to the disease site. For a specific cancer, the monoclonal antibody is the site-selective delivery agent, which binds to the tumor cells, either in the bloodstream or in micro-metastases, and delivers the isotope to the tumor cells.

Chelators are the linking molecules used to attach the alpha particle to the monoclonal antibody. To utilize the alpha emitting isotopes for cancer treatment, a linkage is created between the isotope and the monoclonal antibody.

At-211 is a cyclotron produced radionuclide by virtue of bombardment of a bismuth target with alpha-particles in a cyclotron via the Bi-207 (a, 2n) At-211 nuclear reaction. Isolation from the cyclotron target is routinely performed by means of dry distillation procedures. Few institutions, however, possess a cyclotron of adequate energy range that is capable of producing At-211. At-211 (t1/2=7.2 h) decays through a branched pathway with each branch resulting in the production of an alpha-particle in its decay to stable Pb-207. The alpha particles from At-211 have a mean energy of 6.8 MeV with a mean LET of 97-99 keV Im-1. Because of its relatively long half-life, At-211 labeled constructs can be used even when the targeting molecule does not gain immediate access to tumor cells. Additionally, its daughter, Po-211, emits K X-rays that allow photon counting of samples and external imaging for biodistribution studies. This radionuclide, by virtue of behaving analogously with iodine halogen chemistry, is also not retained as well as other alpha-emitting radiometals post-internalization into cells, which is a factor to be considered.

Bi-212 (t1/2=60.6 min) emits an alpha-particle with a mean energy of 7.8 MeV from the decay of Th-228 to stable Pb-208. A generator that uses Ra-224 as the parent radionuclide provides for on-site production of Bi-212 for radiolabeling targeting vectors, such as monoclonal antibodies, since the half-life is too short for realistic transportation between sites. The Ra-224 actually originates from weapons development and is extracted from Th-229, currently at Pacific Northwest Laboratories with the Th-228 originally being purified from U-232. One daughter from the decay of Bi-212, TI-208, emits a 2.6-MeV c-ray that requires heavy shielding to minimize radiation exposure to personnel, thereby limiting the clinical utility of this radioisotope. However, it is unclear what level of shielding is really necessary in a clinical setting due to the combination of both actual dosing schedules and short half-life. After Bi-212 has been selectively eluted from the ion-exchange resin of the Ra-224 generator either in the form of chloride or the tetraiodide complex, the isotope can be used after pH adjustment to radiolabel monoclonal antibodies, peptides, or other vectors conjugated with a suitable bifunctional chelating agent such as the C-functionalized trans-cyclohexyldiethylenetriamine pentaacetic acid derivative, CHX-DTPA. Both branches involve the emission of an alpha-particle and a beta-particle. Because of this mixture of high- and low-linear-energy-transfer radiation, it is more difficult to attribute observed cytotoxicities directly to alpha-particle-mediated effects. Conversely, the longer range of its beta-particles may help kill cells which otherwise would be spared due to heterogeneous tumour accumulation of a Bi-212 labeled agent. Perhaps the most significant limitation of Bi-212 for radiotherapy is its 60.6 min half-life, which limits its use to settings in which rapid localization in the tumor can be accomplished. Clearly, applications involving intravenous administration of microbubbles, macromolecules, such as monoclonal antibodies, are compatible with the short half-life of Bi-212. An advantage of Bi-212 is that it can be obtained conveniently from a longer-lived. Ra-224 parent in the form of a portable generator.

Pb-212 (t1/2=10.2 h) is actually a beta-emitter and is the immediate parental radionuclide of Bi-212. Its inclusion here is justified since Pb-212 has been evaluated as an in vivo generator for the production of Bi-212 thereby effectively extending the half-life of Bi-212 to ˜11 h. However, during the decay processes, approximately 30% of the formed Bi-212 is released from the chelation environment. Nonetheless, the combination of greater efficacy as compared to Bi-212 on the basis of ICi vs. mCi lowered administered dose, and issues of availability vs. cost, all combined with appropriate usage continue to promote the use of this radionuclide as a viable therapeutic within specific limitations. Pb-212 is available from the same Ra-224 generator that facilitates the production of Bi-212, and may be selectively eluted by controlling the pH of the HCl eluant from that same ion-exchange based generator system vs. Bi-212 for labeling monoclonal antibodies. Concerns regarding the 2.6-MeV gamma-ray from the TI-208 daughter are diminished due to decreased dose levels combined with half-life.

Bi-213 is also available from a very similar generator based technology from its parent radionuclide Ac-225 dispersed onto a cation exchange resin to prevent charring and decomposition of resin due to the confined radiation flux. The source of Ac-225 in the United States is currently limited to Oak Ridge National Laboratories where the source materials extend back to Ra-225 extracted from Th-229 which again has its origin in weapons development from U-233. Bi-213 decays to stable Bi-209 by emitting an alpha-particle and 2 beta--particles. Additionally, a 440-keV photon emission allows biodistribution, pharmacokinetic, and dosimetry studies to be performed. Similarly to Bi-212, after elution from the Ac-225 generator, Bi-213 is readily conjugated to monoclonal antibodies, peptides, or other vectors that have been modified with a suitable bifunctional chelating agent, such as CHX-ADTPA.

Ra-223 (t1/2=11.4 d) can be provided in a generator form from the Ac-227 (t1/2=21.8 y) parent and is also available from uranium mill tailings in large quantities. Similar to Ac-225, Ra-223 ultimately provides for the emission of 4 alpha-particles through its decay scheme and daughters. Because of inherent boneseeking properties, cationic Ra-223 may be a promising candidate for the delivery of high-LET radiation to cancer cells on bone surfaces. A Phase I clinical study demonstrated pain relief and reduction in tumor marker levels in the treatment of skeletal metastases in patients with prostate and breast cancer. Development of chelation chemistry actively targeted Ra-223 continues to be pursued, however, the retention and biological trafficking of the decay process daughters remains a problematic challenge. The first daughter in the Ra-223 decay pathway is Rn-219, a gaseous product that would pose a serious challenge to control in vivo. Thus, the biodistribution and targeting as well as those issues pertaining to control and trafficking of the decay daughters remain under investigation.

Ac-225 (t1/2=10.0 d) decays sequentially by alpha emission through three daughter radionuclides, Fr-221 (t1/2=4.8 min), At-217 (t1/2=32.3 ms), and Bi-213 (t1/2=45.6 min), each of which then also emits an alpha-particle. Ac-225 can be produced by the natural decay of U-233 or by accelerator-based methods. Targeted Ac-225 as a therapeutic, in theory may be as much as about 1000 times more potent than Bi-213 containing analogs by virtue of this alpha particle cascade to a cancer cell. While this increased potency might render Ac-225 more effective than other alpha emitters, the biological fate of the free daughter radioisotopes in circulation after decay of Ac-225 is unresolved; the qualities of the chelation chemistry used to sequester this element in vivo are equally unresolved.

Astatine, the heaviest of the group VIIA elements, has no stable isotopes. At-211 has a half-life of 7.2 h and a strong case can be made that At-211 is the most promising radionuclide for alpha-particle radiotherapy. Each decay of At-211 yields one alpha-particle. The first branch (42%) involves decay to Bi-207 via the emission of 5.87 MeValpha-particles, whereas the second branch (58%) is by electron capture Po-211 with 520 ms half-life, which in turn de-excites by the emission of 7.45 MeV alpha-particles. The lower and higher energy alpha-particles emitted by At-211 have approximate mean ranges in tissue of 55 and 80 μm, respectively. Because of the electron capture decay of At-211 to Po-211, polonium K x-rays also are emitted. These emissions make it convenient to count At-211 activity levels and to perform external imaging of At-211 tissue distributions. The largest impediment to utilizing At-211 for radiotherapy is its lack of availability due to the need for a medium-energy cyclotron with an alpha-particle beam for its production. The standard method for At-211 production is via cyclotron bombardment of natural bismuth metal targets with 28-29 MeV alpha-particles by the Bi-207.alpha; 2n/211 At reaction, followed by isolation of At-211 by dry distillation. Beam energies are kept below the threshold for the (alpha; 3n/At-210 reaction, a product which decays with an 8.1 h half-life to Po-210, an alpha-emitter of 138 day half-life, which must be excluded because of its potential toxicity to normal tissues including bone marrow.

The antibody/chelator complex improves stability and quality. Shortly before clinical use, a precisely prepared single patient dose of antibody/chelator complex will be mixed with freshly prepared Bismuth-213 isotope. The Bismuth-213 is easily and rapidly eluted from Actinium-225. In the hospital laboratory, the Actinium-225 is received as a generator and “milked” to obtain the Bismuth-213. The procedure has been developed and is currently being used in the first clinical trial against Acute Myeloid Leukemia at Memorial Sloan Kettering Cancer Center.

Actinium-225 and Bismuth-213 pose low risk to pharmacy personnel since the alpha particle radiation cannot penetrate the thickness of a pair of disposable plastic gloves. Hence, the facilities and equipment needed for handling alpha particle therapy are minimal compared to other types of radiation used in medical settings.

Radionuclides useful in certain embodiments of the present invention are presented in the table below:

TABLE 1

| ISO- | HALF- | |
|---|---|---|
| TOPE | LIFE | KNOWN APPLICATIONS |
| Ac-225 | 10.0 d | Monoclonal antibody attachment used for cancer |
| | | treatment (RIT), also parent of Bi-213. |
| Ac-227 | 21.8 y | Parent of Ra-223 (Monoclonal antibody attachment |
| | | used for cancer treatment (RIT). |
| Am-241 | 432 y | Osteoporosis detection, heart imaging. |
| As-72 | 26.0 h | Planar imaging, SPECT or PET. |
| As-74 | 17.8 d | Positron-emitting isotope with biomedical |
| | | applications. |
| At-211 | 7.21 h | Monoclonal antibody attachment (alpha emitter) |
| | | used for cancer treatment (RIT), used with F-18 for |
| | | in vivo studies. |
| Au-198 | 2.69 d | Cancer treatment using mini-gun (B), treating |
| | | ovarian, prostate, and brain cancer. |
| B-11 | Stable | Melanoma and brain tumor treatment. |
| Be-7 | 53.2 d | Used in berylliosis studies. |
| Bi-212 | 1.10 h | Monoclonal antibody attachment (alpha emitter) |
| | | used for cancer treatment (RIT), cellular dosimetry |
| | | studies. |
| Bi-213 | 45.6 m | Monoclonal antibody attachment (alpha emitter) |
| | | used for cancer treatment (RIT). |
| Br-75 | 98 m | Planar imaging, SPECT or PET (C). |
| Br-77 | 57 h | Label radiosentizers for Te quantization of hypoxia |
| | | in tumors, and monoclonal antibody labeling. |
| C-11 | 20.3 m | Radiotracer in PET scans to study normal/abnormal |
| | | brain functions. |
| C-14 | 5730 y | Radiolabeling for detection of tumors (breast, et |
| | | al.). |
| Ca-48 | Stable | |
| Cd-109 | 462 d | Cancer detection (C), pediatric imaging (C). |
| Ce-139 | 138 d | Calibrates high-purity germanium gamma detectors. |
| Ce-141 | 32.5 d | Gastrointestinal tract diagnosis, measuring regional |
| | | myocardial blood flow. |
| Cf-252 | 2.64 y | Cervical, melanoma, brain cancer treatment. |
| Co-55 | 17.5 h | Planar imaging, SPECT or PET (B). Used in PET |
| | | imaging of damaged brain tissue after stroke. |
| Co-57 | 272 d | Gamma camera calibration, should be given high |
| | | priority, radiotracer in research and a source for X- |
| | | ray fluorescence spectroscopy. |
| Co-60 | 5.27 y | Teletherapy (destroy cancer cells), disinfect surgical |
| | | equipment and medicines, external radiation cancer |
| | | therapy (E). |
| Cr-51 | 27.7 d | Medical, cell labeling and dosimetry. |
| Cs-130 | 29.2 m | Myocardial localizing agent. |
| Cs-131 | 9.69 d | Intracavity implants for radiotherapy. |
| Cs-137 | 30.2 y | Blood irradiators, PET imaging, tumor treatment. |
| Cu-61 | 3.35 h | Planar imaging, SPECT or PET (B). |
| Cu-62 | 4.7 m | Positron emitting radionuclide (B), cerebral and |
| | | myocardial blood flow used As-a tracer in |
| | | conjunction with Cu 64 (B). |
| Cu-64 | 12.7 h | PET scanning (C), planar imaging (C), SPECT |
| | | imaging (C) dosimetry studies (C), cerebral and |
| | | myocardial blood flow (C), used with Cu-62 (C), |
| | | treating of colorectal cancer. |
| Cu-67 | 61.9 h | Cancer treatment/diagnostics, monoclonal |
| | | antibodies, radioimmunotherapy, planar imaging, |
| | | SPECT or PET. |
| Dy-165 | 2.33 h | Radiation synovectomy, rheumatoid arthritis |
| | | treatment. |
| Eu-152 | 13.4 y | Medical. |
| Eu-155 | 4.73 y | Osteoporosis detection. |
| F-18 | 110 m | Radiotracer for brain studies (C), PET imaging (C). |
| Fe-55 | 2.73 y | Heat source. |
| Fe-59 | 44.5 d | Medical. |
| Ga-64 | 2.63 m | Treatment of pulmonary diseases ending in fibrosis |
| | | of lungs. |
| Ga-67 | 78.3 h | Imaging of abdominal infections (C), detect |
| | | Hodgkins/non-Hodgkins lymphoma (C), used with |
| | | In-111 for soft tissue infections and osteomyelitis |
| | | detection (C), evaluate sarcoidiodis and other |
| | | granulomaous diseases, particularly in lungs and |
| | | mediastiusim (C). |
| Ga-68 | 68.1 m | Study thrombosis and atherosclerosis, PET imaging, |
| | | detection of pancreatic cancer, attenuation |
| | | correction. |
| Gd-153 | 242 d | Dual photon source, osteoporosis detection, SPECT |
| | | imaging. |
| Ge-68 | 271 d | PET imaging. |
| H-3 | 12.3 y | Labeling, PET imaging. |
| I-122 | 3.6 m | Brain blood flow studies. |
| I-123 | 13.1 h | Brain, thyroid, kidney, and myocardial imaging (C), |
| | | cerebral blood flow (ideal for imaging) (C), |
| | | neurological disease (Alzheimer's) (C). |
| I-124 | 4.17 d | Radiotracer used to create images of human thyroid, |
| | | PET imaging. |
| I-125 | 59.9 d | Osteoporosis detection, diagnostic imaging, tracer for |
| | | drugs, monoclonal antibodies, brain cancer |
| | | treatment (I-131 replacement), SPECT imaging, |
| | | radiolabeling, tumor imaging, mapping of receptors |
| | | in the brain (A), interstitial radiation therapy |
| | | (brachytherapy) for treatment of prostate cancer (E). |
| I-131 | 8.04 d | Lymphoid tissue tumor/hyperthyroidism treatment |
| | | (C), antibody labeling (C), brain biochemistry in |
| | | mental illness (C), kidney agent (C), thyroid |
| | | problems (C), alternative to Tl-201 for |
| | | radioimmunotherapy (C), imaging, cellular |
| | | dosimetry, scintigraphy, treatment of graves disease, |
| | | treatment of goiters, SPECT imaging, treatment of |
| | | prostate cancer, treatment of hepatocellular |
| | | carcinoma, treatment of melanoma (A), locate |
| | | osteomyelitis infections (A), radiolabeling (A), |
| | | localize tumors for removal (A), treatment of spinal |
| | | tumor (A), locate metastatic lesions (A), treAt- |
| | | neuroblastoma (A), internal (systemic) radiation |
| | | therapy (E), treatment of carcinoma of the thyroid |
| | | (E). |
| I-132 | 2.28 h | Mapping precise area of brain tumor before |
| | | operating. |
| In-111 | 2.81 d | Detection of heart transplant rejection (C), imaging |
| | | of abdominal infections (C), antibody labeling (C) |
| | | cellular immunology (C), used with Ga-67 for soft |
| | | tissue infection detection and ostemyelitis detection |
| | | (C), concentrates in liver, kidneys (C), high specific |
| | | activity (C), white blood cell imaging, cellular |
| | | dosimetry, myocardial scans, treatment of leukemia, |
| | | imaging tumors. |
| In-115 | 4.49 h | Label blood elements for evaluating inflammatory |
| m | | bowel disease. |
| Ir-191 | 6 s | Cardiovascular angiography. |
| m | | |
| Ir-192 | 73.8 d | Implants or “seeds” for treatment of cancers of the |
| | | prostate, brain, breast, gynecological cancers. |
| Kr-81 | 13.3 s | Lung imaging. |
| m | | |
| Lu-177 | 6.68 d | Heart disease treatment (restenosis therapy), cancer |
| | | therapy. |
| Mn-51 | 46.2 m | Myocardial localizing agent. |
| Mn-52 | 5.59 d | PET scanning. |
| Mo-99 | 65.9 h | Parent for Tc-99 m generator used for brain, liver, |
| | | lungs, heart imaging. |
| N-13 | 9.97 m | PET imaging, myocardial perfusion. |
| Nb-95 | 35 d | Study effects of radioactivity on pregnant women |
| | | and fetus, myocardial tracer, PET imaging. |
| O-15 | 122 s | Water used for tomographic measuring of cerebral |
| | | blood flow (C), PET imaging (C), SPECT imaging. |
| Os-191 | 15.4 d | Parent for Ir-191m generator used for cardiovascular |
| | | angiography. |
| Os-194 | 6.00 y | Monoclonal antibody attachment used for cancer |
| | | treatment (RIT). |
| P-32 | 14.3 d | Polycythaemia Rubra Vera (blood cell disease) and |
| | | leukemia treatment, bone disease |
| | | diagnosis/treatment, SPECT imaging of tumors (A), |
| | | pancreatic cancer treatment (A), radiolabeling (A). |
| P-33 | 25 d | Labeling. |
| Pb-203 | 2.16 d | Planar imaging, SPECT or PET (used with Bi-212) |
| | | (B), monoclonal antibody immunotherapy (B), |
| | | cellular dosimetry. |
| Pb-211 | 36.1 m | Radioactive labeling for thearpy. |
| Pb-212 | 10.6 h | Radioactive label for therapy using antibodies, |
| | | cellular dosimetry. |
| Pd-103 | 17 d | Prostate cancer treatment. |
| Pd-109 | 13.4 h | Potential radiotherapeutic agent. |
| Pu-238 | 2.3 y | Pacemaker (no Pu-236 contaminants). |
| Ra-223 | 11.4 d | Monoclonal antibody attachment (alpha emitter) |
| | | used for cancer treatment (RIT). |
| Ra-226 | 1.60e3 | Target isotope to make Ac-227, Th-228, Th-229 |
| | y | (Parents of alpha emitters used for RIT). |
| Rb-82 | 1.27 m | Myocardial imaging agent, early detection of |
| | | coronary artery disease, PET imaging, blood flow |
| | | tracers. |
| Re-186 | 3.9 d | Cancer treatment/diagnostics, monoclonal |
| | | antibodies, bone cancer pain relief, treatment of |
| | | rheumatoid arthritis, treatment of prostate cancer, |
| | | treating bone pain. |
| Re-188 | 17 h | Monoclonal antibodies, cancer treatment. |
| Rh-105 | 35.4 h | Potential therapeutic applications: target neoplastic |
| | | cells (e.g., small cell lung cancer) (A), labeling of |
| | | molecules and monoclonal antibodies (A). |
| Ru-97 | 2.89 d | Monoclonal antibodies label (C), planar imaging (C), |
| | | SPECT or PET techniques (C), gamma-camera |
| | | imaging. |
| Ru-103 | 39 d | Myocardial blood flow, radiolabeling microspheres, |
| | | PET imaging. |
| S-35 | 87.2 d | Nucleic acid labeling, P-32 replacement, cellular |
| | | dosimetry. |
| Sc-46 | 84 d | Regional blood flow studies, PET imaging. |
| Sc-47 | 3.34 d | Cancer treatment/diagnostics (F), monoclonal |
| | | antibodies (F), radioimmunotherapy (F). |
| Se-72 | 8.4 d | Brain imaging, generator system with As-72, |
| | | monoclonal antibody immunotherapy. |
| Se-75 | 120 d | Radiotracer used in brain studies, scintigraphy |
| | | scanning. |
| Si-28 | Stable | Radiation therapy of cancer. |
| Sm-145 | 340 d | Brain cancer treatment using I-127 (D). |
| Sm-153 | 2.00 d | Cancer treatment/diagnostics (C), monoclonal |
| | | antibodies (C), bone cancer pain relief (C), higher |
| | | uptake in diseased bone than Re-186 (C), treatment |
| | | of leukemia. |
| Sn- | 13.6 d | Bone cancer pain relief. |
| 117m | | |
| Sr-85 | 65.0 d | Detection of focal bone lesions, brain scans. |
| Sr-89 | 50 d | Bone cancer pain palliation (improves the quality of |
| | | life), cellular dosimetry, treatment of prostate |
| | | cancer, treatment of multiple myeloma, osteoblastic |
| | | therapy, potential agent for treatment of bone |
| | | metastases from prostate and breast cancer (E). |
| Sr-90 | 29.1 y | Generator system with Y-90 (B), monoclonal |
| | | antibody immunotherapy (B). |
| Ta-178 | 9.3 m | Radionuclide injected into patients to allow viewing |
| | | of heart and blood vessels. |
| Ta-179 | 1.8 y | X-ray fluorescence source and in thickness gauging |
| | | (might be a good substitute for Am-241). |
| Ta-182 | 115 d | Bladder cancer treatment, internal implants. |
| Tb-149 | 4.13 h | Monoclonal antibody attachment used for cancer |
| | | treatment (RIT). |
| Tc-96 | 4.3 d | Animal studies with Tc-99m. |
| Tc-99m | 6.01 h | Brain, heart, liver (gastoenterology), lungs, bones, |
| | | thyroid, and kidney imaging (C), regional cerebral |
| | | blood flow (C), equine nuclear imaging (C), |
| | | antibodies (C), red blood cells (C), replacement for |
| | | Tl-201 (C). |
| Th-228 | 720 d | Cancer treatment, monoclonal antibodies, parent of |
| | | Bi-212. |
| Th-229 | 7300 y | Grandparent for alpha emitter (Bi-213) used for |
| | | cancer treatment (RIT), parent of Ac-225. |
| Tl-201 | 73.1 h | Clinical cardiology (C), heart imaging (C), less |
| | | desirable nuclear characteristics than Tc-99m for |
| | | planar and SPECT imaging (C), myocardial perfusion, |
| | | cellular dosimetry. |
| Tm-170 | 129 d | Portable blood irradiations for leukemia, lymphoma |
| | | treatment, power source. |
| Tm-171 | 1.9 y | Medical. |
| W-188 | 69.4 d | Cancer treatment, monoclonal antibodies, parent for |
| | | Re-188 generator. |
| Xe-127 | 36.4 d | Neuroimaging for brain disorders, research for |
| | | variety of neuropsychiatric disorders, especially |
| | | schizophrenia and dementia, higher resolution SPECT |
| | | studies with lower patient dose, lung imaging (some |
| | | experts believe it is superior to Xe-133 in inhalation |
| | | lung studies). |
| Xe-133 | 5.25 d | Lung imaging (C), regional cerebral blood flow (C), |
| | | liver imaging (gas inhalation) (C), SPECT imaging of |
| | | brain, lung scanning, lesion detection. |
| Y-88 | 107 d | Substituted for Y-90 in development of cancer tumor |
| | | therapy. |
| Y-90 | 64 h | Internal radiation therapy of liver cancer (C), |
| | | monoclonal antibodies (C), Hodgkins disease, and |
| | | hepatoma (C), cellular dosimetry, treating |
| | | rheumatoid arthritis, treating breast cancer, |
| | | treatment of gastrointestinal adenocarcinomas (A). |
| Y-91 | 58.5 d | Cancer treatment (RIT), cellular dosimetry. |
| Yb-169 | 32 d | Gastrointestinal tract diagnosis. |
| Zn-62 | 9.22 h | Parent of Cu-62, a positron-emitter, used for the |
| | | study of cerebral and myocardial blood flow. |
| Zn-65 | 244 d | Medical. |
| Zr-95 | 64.0 d | Medical. |

Microparticles and microbubbles can be used in medical applications, such as imaging. Microbubbles can be formed as spray dried microspheres, such as those made using proteins or other biocompatible materials. Some proteins for forming microbubbles include heat-denaturable biocompatible proteins, such as, for example, albumin, hemoglobin, and/or collagen. Microbubbles may be stabilized by surfactants, lipids, proteins, lipoproteins, polymers, and/or polysaccharides.

Nanoparticles are also within the scope of certain embodiments of the present invention. Nanoparticles may be formed from a variety of materials, including metal, intermetallics, and organic materials. Nanoparticles are characterized by dimensions in the submicron ranges and can exhibit properties unique to their size. That is, the properties of a nanoparticle may be different from that of the same material in bulk form. Core-shell nanoparticles and liposome-based nanoparticles are particularly useful examples of nanoparticles for certain embodiments of the invention. Further, ultrasound has been shown to drive nanoparticles into cells. These particles can also be used to deliver a payload that is not activated, simply a form of passive delivery. The term “microparticles” used herein includes, but is not limited to, microparticles, microbubbles and nanoparticles.

Contrast-enhanced ultrasound (CEUS) is the application of ultrasound contrast agents to traditional medical sonography. Ultrasound contrast agents are gas-filled microbubbles that are administered intravenously to the systemic circulation. Microbubbles have a high degree of echogenicity, which is the ability of an object to reflect the ultrasound waves. The echogenicity difference between the gas in the microbubbles and the soft tissue surroundings of the body is immense. Thus, ultrasonic imaging using microbubble contrast agents enhances the ultrasound backscatter, or reflection of the ultrasound waves, to produce a unique sonogram with increased contrast due to the high echogenicity difference. Contrast-enhanced ultrasound can be used to image blood perfusion in organs, measure blood flow rate in the heart and other organs, and has other applications as well.

Targeting ligands that bind to receptors characteristic of intravascular diseases can be conjugated to microbubbles, enabling the microbubble complex to accumulate selectively in areas of interest, such as diseased or abnormal tissues. This form of molecular imaging, known as targeted contrast-enhanced ultrasound, will generate a strong ultrasound signal if targeted microbubbles bind in the area of interest. Targeted contrast-enhanced ultrasound can potentially have many applications in both medical diagnostics and medical therapeutics.

There are a variety of microbubbles contrast agents. Microbubbles differ in their shell makeup, gas core makeup, and whether or not they are targeted.

Selection of shell material determines how easily the microbubble is taken up by the immune system. A more hydrophilic material tends to be taken up more easily, which reduces the microbubble residence time in the circulation. This reduces the time available for contrast imaging. The shell material also affects microbubble mechanical elasticity. The more elastic the material, the more acoustic energy it can withstand before bursting. Currently, microbubble shells are composed of albumin, galactose, lipid, or polymers.

The microbubble gas core is an important part of the ultrasound contrast microbubble because it determines the echogenicity. When gas bubbles are caught in an ultrasonic frequency field, they compress, oscillate, and reflect a characteristic echo-this generates the strong and unique sonogram in contrast-enhanced ultrasound. Gas cores can be composed of air, or heavy gases like perfluorocarbon, or nitrogen. Heavy gases are less water-soluble so they are less likely to leak out from the microbubble to impair echogenicity. Therefore, microbubbles with heavy gas cores are likely to last longer in circulation.

Optison, a Food and Drug Administration (FDA)-approved microbubble made by GE Healthcare, has an albumin shell and octafluoropropane gas core.

Definity, a Food and Drug Administration (FDA)-approved microbubble made by Lantheus Medical Imaging, has an lipid shell and octafluoropropane gas core.

Targeted microbubbles retain the same general features as untargeted microbubbles, but they are outfitted with ligands that bind specific receptors expressed by cell types of interest, such as inflamed cells or cancer cells. Current microbubbles in development are composed of a lipid monolayer shell with a perfluorocarbon gas core. The lipid shell is also covered with a polyethylene glycol (PEG) layer. PEG prevents microbubble aggregation and makes the microbubble more non-reactive. It temporarily “hides” the microbubble from the immune system uptake, increasing the amount of circulation time, and hence, imaging time. In addition to the PEG layer, the shell is modified with molecules that allow for the attachment of ligands that bind certain receptors. These ligands are attached to the microbubbles using carbodiimide, maleimide, or biotin-streptavidin coupling. Biotin-streptavidin is the most popular coupling strategy because biotin's affinity for streptavidin is very strong and it is easy to label the ligands with biotin. Currently, these ligands are monoclonal antibodies produced from animal cell cultures that bind specifically to receptors and molecules expressed by the target cell type. Since the antibodies are not humanized, they will elicit an immune response when used in human therapy. Humanizing antibodies is an expensive and time-intensive process, so it would be ideal to find an alternative source of ligands, such as synthetically manufactured targeting peptides that perform the same function, but without the immune issues.

There are two forms of contrast-enhanced ultrasound, untargeted and targeted. The two methods slightly differ from each other.

Untargeted microbubbles, such as the aforementioned Optison or Definity, are injected intravenously into the systemic circulation in a small bolus. The microbubbles will remain in the systemic circulation for a certain period of time. During that time, ultrasound waves are directed on the area of interest. When microbubbles in the blood flow past the imaging window, the microbubbles' compressible gas cores oscillate in response to the high frequency sonic energy field, as described in the ultrasound article. The microbubbles reflect a unique echo that stands in stark contrast to the surrounding tissue due to the orders of magnitude mismatch between microbubble and tissue echogenicity. The ultrasound system converts the strong echogenicity into a contrast-enhanced image of the area of interest. In this way, the bloodstream's echo is enhanced, thus allowing the clinician to distinguish blood from surrounding tissues.

Targeted contrast-enhanced ultrasound works in a similar fashion, with a few alterations. Microbubbles targeted with ligands that bind certain molecular markers that are expressed by the area of imaging interest are still injected systemically in a small bolus. Microbubbles travel through the circulatory system, eventually finding their respective targets and binding specifically. Ultrasound waves can then be directed on the area of interest. If a sufficient number of microbubbles have bound in the area, their compressible gas cores oscillate in response to the high frequency sonic energy field, as described in the ultrasound article. The targeted microbubbles also reflect a unique echo that stands in stark contrast to the surrounding tissue due to the orders of magnitude mismatch between microbubble and tissue echogenicity. The ultrasound system converts the strong echogenicity into a contrast-enhanced image of the area of interest, revealing the location of the bound microbubbles. Detection of bound microbubbles may then show that the area of interest is expressing that particular molecular, which can be indicative of a certain disease state, or identify particular cells in the area of interest.

Untargeted contrast-enhanced ultrasound is currently applied in echocardiography. Targeted contrast-enhanced ultrasound is being developed for a variety of medical applications. Untargeted microbubbles like Optison and Definity are currently used in echocardiography.

Microbubbles can enhance the contrast at the interface between the tissue and blood and provide a method for organ edge delineation. A clearer picture of this interface gives the clinician a better picture of the structure of an organ. Tissue structure is crucial in echocardiograms, where a thinning, thickening, or irregularity in the heart wall indicates a serious heart condition that requires either monitoring or treatment.

Contrast-enhanced ultrasound holds the promise for (1) evaluating the degree of blood perfusion in an organ or area of interest and (2) evaluating the blood volume in an organ or area of interest. When used in conjunction with doppler ultrasound, microbubbles can measure myocardial flow rate to diagnose valve problems. The relative intensity of the microbubble echoes can also provide a quantitative estimate on blood volume.

In inflammatory diseases such as Crohn's disease, atherosclerosis, and even heart attacks, the inflamed blood vessels specifically express certain receptors like VCAM-1, ICAM-1, E-selectin. If microbubbles are targeted with ligands that bind these molecules, they can be used in contrast echocardiography to detect the onset of inflammation. Early detection allows the design of better treatments.

There has been an increasing interest in the biomedical research community to enhance the adhesion efficiency of microbubble contrast agents in order to realize targeted contrast-enhanced ultrasound's immense diagnostic and therapeutic potentials. Microbubbles with monoclonal antibodies that bind endothelial markers of inflammation, specifically the cell adhesion molecules P-selectin, ICAM-1, and VCAM-1 showed that these complexes enable targeted ultrasound imaging of inflammation. But, the aforementioned efficiency of microbubble adhesion to the molecular target was poor and a large fraction of microbubbles that bound to the target rapidly detached, especially at high shear stresses of physiological relevance. Effective contrast-enhanced ultrasound requires efficient microbubble binding at the area of imaging interest.

Leukocytes possess high adhesion efficiencies, partly due to a dual-ligand selectin-integrin cell arrest system. One ligand:receptor pair (PSGL-1:selectin) has a fast bond on-rate to slow the leukocyte and allows the second pair (integrin:immunoglobulin superfamily), which has a slower on-rate but slow off-rate to arrest the leukocyte, kinetically enhancing adhesion. Dual-ligand targeting of distinct receptors to polymer microspheres for drug delivery can promote an increase in microsphere binding. Microbubbles targeted to bind two distinct receptors can have increased microbubble adhesion strength. Biomimicry of the leukocyte's selectin-integrin cell arrest system can improve microbubble adhesion efficiency.

Contrast-enhanced ultrasound adds these additional advantages: (1) The body is 90% water, and therefore, acoustically homogeneous. Blood and surrounding tissues have similar echogenicities, so it is also difficult to clearly discern the degree of blood flow, perfusion, or the interface between the tissue and blood using traditional ultrasound; (2) Ultrasound imaging allows real-time evaluation of blood flow; (3) Ultrasonic molecular imaging is safer than molecular imaging modalities such as radionuclide imaging because it does not involve radiation; (4) Alternative molecular imaging modalities, such as MRI, PET, and SPECT are very costly. Ultrasound, on the other hand, is very cost-efficient and widely available; (5) Since microbubbles can generate such strong signals, a lower intravenous dosage is needed, micrograms of microbubbles are needed compared to milligrams for other molecular imaging modalities such as MRI contrast agents; and (6) Targeting strategies for microbubbles are versatile and modular. Targeting a new area only entails conjugating a new ligand.

Delivery of alpha particle radiation via microbubbles presents certain advantages, including (1) Short range (5 cell diameters) and great power (30× greater than beta-particles) of the alpha particles effectively kills tumor cells; (2) The short range, short half life and lack of residual radiation limits collateral damage to neighboring normal tissues. Animal and clinical studies to date show significant damage to normal tissues in proximity to the alpha-particle irradiation; (3) Radionuclides, cancer specific antibodies, and MRI or radionuclear markers for imaging can be attached to the microbubble in any combination; (4) Microbubbles are the same size as red blood cells, so they flow freely in blood vessels of all size, and they are safely disposed of by the body; (5) The problem of access to the tumors is eliminated since the microbubble-radionuclides go through all size blood vessels including capillaries; (6) The uniform distribution of the microbubbles, and hence the attached alpha-particles, assures predictable dosimetry; (7) Sonication causes precise, localized delivery of the radioactive material resulting in a high concentration within the tumor; (8) Whole body sonication would be expected to irradiate every cell in the field that has a blood supply or is near to a blood vessel, and normal cells should be little affected while every cancer cell, including metastases, should be susceptible; (9) Microbubbles such as Optison have been approved for clinical use; (10) Alpha-particles have been approved for clinical use; (11) Echocardiographic equipment is much less expensive than is the equipment used in external beam irradiation; and (12) The isotope can be prepared at the bedside.

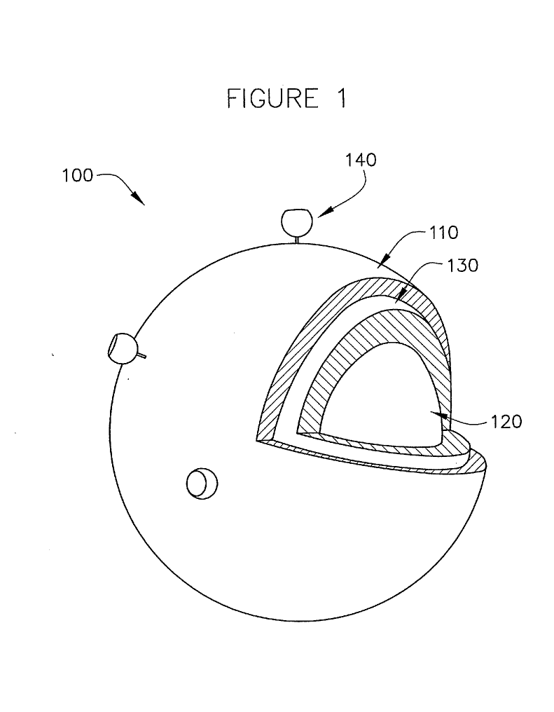

Referring now to FIG. 1, a targeted microbubble according to certain embodiments of the present invention comprises a lipid bubble 110. Within lipid bubble 110 is gas 120 and therapeutic agent 130. Therapeutic agent 130 comprises at least a radionuclide, and can optionally include a ligand or an antibody. Surface agent 140, which is attached to the surface of lipid bubble 110, includes a therapeutic agent, such as a radionuclide, a ligand, and an antibody, or combinations thereof.

In certain embodiments of the present invention, the imaging marker and the therapeutic radionuclide are both attached to the same microbubble, so the distribution of imaging agents should be identical to the distribution of therapeutic agents. Advantageously, this allows for precise dosimetery and other benefits. As is discussed elsewhere, technetium or other radionuclear markers as well as MRI markers may also be attached to the same microbubble that carries the therapeutic alpha-emitter and/or a tumor specific ligand].

Alpha-emitters can be used alone or in combination with a cancer specific antibody as attachments to the microbubbles. Of note, Optison, and certain alpha-emitters (including Bismuth 213) have already been approved by the FDA. Some cancer-specific antibodies have also been approved. Nuclear and MRI imaging markers can also be attached to the microbubble in combination with the therapeutic alpha-emitter and the cancer specific antibody. In addition, certain drugs and radiosensitizers can also be attached.

Microbubbles are capable of delivering drugs to specific tissue. Microbubbles can be loaded with radioisotope payloads (e.g., alpha emitters) and injected into a vein, followed by localized ultrasound. In this way, microbubbles are a specific and local delivery is controlled by the local application of ultrasound. However, as currently used, microbubbles are relatively stable and circulate through the whole body, delivery of material could partly result in deposition of the contents of the microbubble in tissue that is not the target tissue, e.g. in the chest wall, or in the lungs, in which microbubbles with higher diameters are filtered. Advantageously, using alpha emitters would minimize side effects because of the short half-life and path length.

Cancer cells also express a specific set of receptors, mainly receptors that encourage angiogenesis, or the growth of new blood vessels. If microbubbles are targeted with ligands that bind receptors like VEGF, they can non-invasively and specifically identify areas of cancers.

Vector DNA can be conjugated to the microbubbles. Microbubbles can be targeted with ligands that bind to receptors expressed by the cell type of interest. When the targeted microbubble accumulates at the cell surface with its DNA payload, ultrasound can be used to burst the microbubble. The force associated with the bursting may temporarily permeablize surrounding tissues and allow the DNA to more easily enter the cells.

Drugs can be incorporated into the microbubble's lipid shell. The microbubble's large size relative to other drug delivery vehicles like liposomes may allow a greater amount of drug to be delivered per vehicle. By targeting the drug-loaded microbubble with ligands that bind to a specific cell type, the microbubble will not only deliver the drug specifically, but can also provide verification that the drug is delivered if the area is imaged using ultrasound.

Microbubbles can be used in various contrast-enhanced ultrasound applications, as shown above. The area of greatest area of promise and growth lies in targeted contrast-enhanced ultrasound. Current microbubble targeting strategies produce low adhesion efficiencies at high vessel shear stresses of physiological relevance. This means that only a small fraction of microbubbles injected into the test subject actually binds to the molecular markers of interest (Takalkar et al., 2004). This is one of the main issues preventing targeted contrast-enhanced ultrasound's jump from bench to bedside.

Combination of imaging and radiotherapy yields superior results: lower doses, lower exposure times, disease specific therapy. MRI markers, nuclear imaging, positron emitters, Single Photon Emission Computed Tomography and echocardiography/sonography.

Diagnostic imaging materials useful in certain embodiments of the present invention include: Indium-111, lodine-123, Copper-62, Copper-64, Gallium-67, Gallium-68, Fluorine-18, Strontium-82, Rubidium-82, Molybdenum-99, Technetium-99m, Thalium-201, Carbon-11, Cesium-137, Chromium-51, Cobalt-57, Cobalt-58, Cobalt-60, Iodine-125, Iodine-131, Krypton-81 m, Nitrogen-13, Oxygen-15, Samarium-153, Strontium-89, Xenon-127, and Ytterbium-169.

Other treatments, such as surgery, chemotherapy, or hormone therapy, may be used in combination with radiation therapy.

The radiotherapy of embodiments of the present invention may be used in conjunction with other therapeutic agents, including stem cells, precursor cells, insulin-producing beta cells, chemotherapeutic agents: hormone antagonists, plant alkaloids, alkylating agents, nitrogen mustard, antibodies, antimetabolites, antitumor antibiotics, anti-angiogenic molecules.

The success of radiolabeling will require effective chemistry for attaching the radionuclide to the microbubble, ligand, antibody and etc. Therefore, a concerted effort has been directed toward the design of chelating agents capable of holding the desired alpha-emitting radionuclide, both selectively and with high stability, to the microbubble, ligand, antibody and etc. This stability must be maintained in the body under physiological conditions and challenged by metal cations (at much higher concentration) that might otherwise compete for binding with the chelate. Bifiunctional chelating agents such as tetraaza macrocycles have been used for this purpose to specifically bind the beta-emitters Y-90 and Cu-67 to antibodies. One of the alpha-emitting radionuclides considered suitable for radioimmunotherapy of cancer is the 11.4 d half-life Ra-223, which decays through a rapid chain of daughter products to Pb-207, emitting four alpha particles, two beta particles, and several gamma rays, with a combined energy of about 28 MeV.

Based on novel chemistries that have been developed over the years, chelating agents that form stable complexes with radionuclides are now available as bifunctional agents. Tumor resistance due to rapid degradation of immunoconjugates and expulsion of isotope metabolites can be overcome by the use of novel conjugation techniques or by therapy with radiometals, which are better retained within the tumor cell after the immunoconjugate has been catabolized. Improvements have been made with the use of chelators to trap free radioactivity and with the use of more stable chelating agents. With the chelating agents 1,4,7,10-tetraazacyclododecane-tetraacetica cid (DOTA). DOTA and diethylene triamine penta-acetate (DTPA), Y-90 has been stably bound to monoclonal antibodies and has demonstrated higher tumor-to-liver and tumor-to-bone ratios. Linkers containing thiourea, thoether, peptide, ester, and disulfide groups were compared for their biodistribution in healthy mice. A disulfide linker led to particularly rapid clearance of radionuclide from the liver and from the whole body. Radioactive antigen-binding proteins have been recombinantly produced by the fusion of antibody genes to physiologic metal chelators such as metallothionein. Antibody-metallothionein conjugates have been shown to be efficient and stable chelators of isotopes such as Tc-99m and In-i 11. Alternatively, other investigators have relied on the fusion of the scFv C-terminal to a peptide that could coordinate radionuclides. Studies in animal models support the usefulness of such systems for diagnostic imaging, as well as their potential for RIT.

Chelators or chelating agents: DTPA (Diethylene triamine pentaacetic acid), DOTA, EDTA (ethylenediaminetetraacetic acid), DOTMA, DOTAP, DOSPA, NOTA, TBBCDA, TETMA, TTHA, TBTC, HBHS, HBED, DMRIE, PDTA, LICAM, MECAM.

Ovarian carcinoma is one example of a disease treated by radiotherapy. Ovarian carcinoma has the highest mortality rate of any gynecological cancer. This is predominantly due to late detection, in particular, the spread of the disease beyond the pelvis by the time of diagnosis. Cytoreductive surgery and systemic therapy have improved the overall survival of these patients; however, even after apparent complete remission, relapses occur secondary to undetected peritoneal spread. Although the initial treatment of late-stage ovarian carcinoma with multiple chemotherapy agents yields response rates of 90%, after 5 years only approximately 20% of patients are reported to be alive. Current salvage strategies include intraperitoneal chemotherapy or abdominopelvic external beam radiotherapy; however, neither of these is of proven value. Intraperitoneal administration of radiocolloids (P-32), a beta- and gamma-emitter, has been explored as an alternative means of delivering higher radiation doses to the peritoneal cavity. Its effectiveness is limited in the treatment of later stage ovarian carcinoma, however, a likely result of its nonuniform distribution within the peritoneum. Moreover, the use of P-32 is associated with various undesirable side effects, most notably small bowel obstruction. Experimental experience with Bismuth 212, an alpha-emitter, indicates that normal tissues were not affected. Ovarian carcinoma is one disease in which patients may benefit from the use of embodiments and methods of the present invention.

Vulnerable plaque is an example of a disease in which patients may benefit from the use of embodiments and methods of the present invention. Vulnerable plaque is a pool of lipids and other components in the wall of an artery covered plaque by a typically fibrous cap. The plaque is termed “vulnerable” because the thin cap is susceptible to breakage or rupture, which can dump the lipid pool into the blood stream. The result of such a rupture is often a major adverse event such as heart attack or stroke.

For the treatment of vulnerable plaque, certain embodiments of the present invention may be targeted to aggregate at or near the plaque. The alpha emitters carried by microparticles can cause a local sclerosis, which would strengthen the thin fibrous cap. The strengthened fibrous cap would be less susceptible to rupture. While it is possible to target microparticles to the wall of the artery through the main lumen of the artery itself, in certain embodiments it is preferred to deliver microparticles to the vaso vasorum. The vaso vasorum is a network of small blood vessels within the wall of an artery. By targeting microparticles to the vaso vasorum, the microparticle may reside locally near the vulnerable plaque longer than if they were the main lumen of the artery.

Chronic synovitis, inflammatory arthritis, rheumatoid arthritis and progressive arthropathy are examples of diseases in which patients may benefit from the use of embodiments and methods of the present invention. These diseases share a common pathology of inflammation of the synovium, a thin layer of tissue that lines joint space. Inflamed synovium triggers cellular proliferation, an increase in blood vessels in the joint space, and fluid secretion. These effects result in chronic swelling of the joint space.

Embodiments of the present invention may be used to treat synoval inflammation. Radiosynovectomy is a procedure in which radioactive substances are injected into the joint space. The radionuclide destroys the proliferating tissue, stops the secretion of fluid, and causes fibrosis in the joint space, effectively sealing the synovium and preventing further swelling. The radioactive substances are typically beta emitting radionuclides and are often administered in colloidal form. As previously described, alpha emitters have some advantages over beta emitters. For example, the effectiveness of alpha emitters is much less dependent on dose rate than is that of beta emitters. Embodiments of the present invention offer advantages over the conventional beta emitter therapy, including the ability to precisely target and control the dose. As with other embodiments described herein, targeting can be accomplished via modification of the surface of a microparticle and may be augmented with imaging means.

Other applications of embodiments of the present invention include the use of microbe-specific monoclonal antibody 18B7 which binds to capsular polysaccharides of the human pathogenic fungus Cryptococcus neoformans. When radiolabeled with Bi-213, biofilm metabolic activity was reduced to 50% while unlabeled 18B7, Bi-213 labeled non-specific monoclonal antibodies, and gamma- and beta-radiation failed to have an effect. Targeted alpha-therapy is an option for the prevention or treatment of microbial biofilms on indwelling medical devices and for infectious diseases for several fungal and bacterial infections.

### EXAMPLES

Experimental Protocol for the Purification of Bi-213

(1) Remove 5 mCi sample of 225Ac+ daughters in Pb pig from packing container and transfer to hood. Remove vial from Pb pig, assess direct radiation dose, and determine with assistance from Health Physics if special handling or shielding requirements beyond ALARA are required. (NOTE: no special handling or shielding requirements are anticipated beyond ALARA and good radioactive laboratory practices.)

(2) Transfer the 5 mCi contents of 225Ac +daughters from shipping vial to a 20 mL liquid scintillation (LSC) vial with two 0.90 mL aliquots of 0.10 M HCl. Transfer 10 mCi aliquot to standard polypropylene g-counting vial, cap, place into a 50 mL centrifuge tube (serving as a secondary container), and prepare for shipment with purified 213Bi (see Step (11) below).

(3) Transfer 0.300 mL of 225Ac +daughters in 0.10 M HCl to column reservoir of a 0.50 mL bed volume (BV) of alkylphosphonate extraction chromatographic column. Column eluate is directed to a 20 mL LSC vial as waste vessel.

(4) Transfer 1.4 mL of 225Ac +daughters in 0.10 M HCl to column reservoir, elute on the 0.50 mL BV of alkylphosphonate extraction chromatographic resin in Step (3), and collect eluate in a 20 mL LSC vial as storage vessel. Step (4) is anticipated to take 10 minutes.

(5) Transfer 0.300 mL of 0.10 M HCl to reservoir of alkylphosphonate extraction chromatographic column and collect eluate into 225Ac storage vessel in Step (4).

(6) Transfer 1.2 mL 0.10 M HCl to reservoir of alkylphosphonate extraction chromatographic resin and direct eluate to waste vessel. Step (6) is anticipated to take 10 minutes.

(7) Transfer 0.300 mL of 0.75 M NaCl in 0.50 M (Na,H)OAc at pH=4.0 (OAc=acetate) to reservoir of alkylphosphonate extraction chromatographic column and direct eluate to waste.

(8) Align alkylphosphonate extraction chromatographic column to drip into the reservoir of a 0.50 mL BV of ion exchange resin.

(9) Transfer 1.5 mL of 0.75 M NaCl in 0.50 M (Na,H)OAc at pH=4.0 to reservoir of alkylphosphonate extraction chromatographic column and direct the purified 213Bi eluate from the ion exchange resin column to a 20 mL LSC vial collection vessel. Step (9) is anticipated to take 10 minutes.

(10) Aliquot 10 mCi of purified 213Bi from Step (9) to a standard polypropylene g-counting vial, cap, and place into a 50 mL centrifuge tube (serving as a secondary container).

(11) Secure all containers with activity behind Pb bricks in hood for overnight storage.

(12) Rinse very trace residual activity from alkylphosponate extraction chromatographic column using 0.10 M HCl and direct eluate to waste. Seal column for storage in hood.

(13) Rinse very trace residual activity from ion exchange resin chromatographic column using 0.75 M NaCl in 0.50 M (Na,H)OAc at pH=4.0 and direct eluate to waste. Seal column for storage in hood.

Radiolabeling of MUC-1 antibody with Bi-213

1) Concentration of conjugate:

Start with 0.3 mg of CHX-A″-DTPA C595 anti-MUC-1 antibody. 25 μL of the antibody was added to 3.0 mL of the generator eluate. Also, 0.1 mL of 150 g/L ascorbic acid, 0.15 mL of 3 M ammonium acetate and 0.1 mL of 0.1 M EDTA were added during the conjugation reaction. The radioconjugate was purified using a P6 column yielding the final Bi-213-antibody product in 8.25 mL of solution (5.0 mL of 0.1% HSA used to elute the Bi-213conjugate from the P6 column)

Molecular weight of MUC-1: 265-400 kDa

So, 25 μL of the antibody would correspond to 0.05 mg in 8.25 mL, which would yield a final concentration of 1.5-2.3×10⁻⁸ moles/L of the antibody.

2) The solution in which the final Bi-213/antibody conjugate is dissolved depends on the generator system:
3.0 mL of 0.75 M NaCl in 0.25 M (Na,H)OAc, pH=4.0
0.1 mL of 150 g/L ascorbic acid
0.15 mL of 3.0 M ammonium acetate
0.1 mL of 0.1 M EDTA
5.0 mL of 0.1% HSA in physiological saline
25 μL of antibody solution (0.3 mg/150 μL)
3.0 mL of 0.1 M HCl+0.1 M NaI
0.1 mL of 150 g/L ascorbic acid
0.3 mL of 3.0 M ammonium acetate
0.1 mL of 0.1 M EDTA
5.0 mL of 0.1% HSA in physiological saline
25 μL of antibody solution (0.3 mg/150 μL)

3)Chelate type: CHX-A″-DTPA, 3 molecules of chelator per molecule of antibody

4) Recommended storage for MUC-1 is 40C. At 2-8° C. the antibody should be stable for 24 months

5) pH must be controlled to prevent Bi hydrolysis and allow conjugation (higher pH's favor conjugation by the DTPA chelator, but also promote Bi hydrolysis)

Trace amounts of some salts (Fe, Al, Ca, etc. . . . ) could interfere with conjugation, given the very low concentration of the chelator-antibody.

1. A composition for the treatment of disease, comprising:
a microparticle having an outer surface;
a targeting agent linked to the outer surface of the microparticle; and
at least one alpha emitting radionuclide carried by the microparticle.

2. The composition of claim 1, wherein at least one alpha emitting radionuclide is contained at least partially within the microparticle.

3. The composition of claim 1, wherein at least one alpha emitting radionuclide is linked to the outer surface of the microparticle.

4. The composition of claim 1, further comprising an echogenic gas within the microparticle.

5. The composition of claim 1, wherein the targeting agent is an antibody.

6. The composition of claim 3, wherein the antibody is a tumor recognizing antibody.

7. The composition of claim 1, further comprising a therapeutic agent carried by the microparticle.

8 The composition of claim 7, wherein the therapeutic agent is a cancer chemotherapeutic agent.

9. The composition of claim 7, wherein the therapeutic agent is selected from the group consisting of hormone antagonists, plant alkaloids, alkylating agents, nitrogen mustard, antibodies, antimetabolites, antitumor antibiotics, antiangiogenesis molecules and combinations thereof.

10. The composition of claim 1, further comprising a radiosensitizer.

11. The composition of claim 1, further comprising an imaging marker.

12. The composition of claim 11, wherein the imaging marker is a radionuclear, magnetic resonance, PET, or SPECT imaging marker.

13. A method for the treatment of disease, comprising:
delivering a microparticle to a treatment site of a patient, the microparticle having a targeting agent linked to an outer surface of the microparticle and the microparticle carrying at least one alpha radiation emitting radionuclide.

14. The method according to claim 13, further comprising applying ultrasound energy to the treatment site.

15. The method according to claim 13, further comprising determining the location of the microparticle using an imaging modality matched to an imaging marker carried by the microparticle.

16. The method according to claim 13 wherein the disease is cancer, vulnerable plaque, or chronic synovitis.

17. A method for the local treatment of a disease in a patient, comprising:
delivering a composition of microparticles to the patient, the microparticles having a targeting agent linked to an outer surface of the microparticle and the microparticle carrying at least one alpha radiation emitting radionuclide and an imaging marker;
locating microparticles near a local treatment site of a patient using an imaging modality; and
applying ultrasound energy to the local treatment site when the microparticles are located near the local treatment site.

18. The method of claim 17 wherein the disease is cancer and the local treatment site is a tumor.

19. The method of claim 17 wherein the disease is vulnerable plaque and the treatment site is the vaso vasorum.

20. The method of claim 17 wherein the disease is chronic synovitis and the treatment site is synovial fluid.
